(19) United States
(12) Patent Application Publication
(10) Pub. No.: US 2026/0216285 A1
(43) Pub. Date: Jul. 30, 2026

(54) METHODS FOR TREATING DISEASES ASSOCIATED WITH CELLULAR SENESCENCE

(71) Applicant: UNIVERSITY OF VIRGINIA PATENT FOUNDATION, Charlottesville, VA (US)

(72) Inventor: Zhiyi Zuo, Charlottesville, VA (US)

(21) Appl. No.: 19/397,488

(22) Filed: Nov. 21, 2025

Related U.S. Application Data
(60) Provisional application No. 63/749,354, filed on Jan. 24, 2025.

(51) Int. Cl. A61K 38/17; A61K 48/00; A61P 43/00; C12N 15/85

(52) U.S. Cl. CPC A61K 38/1709; A61K 48/005; A61P 43/00; C12N 15/85

(57) ABSTRACT

Methods for treating diseases associated with cellular senescence and/or age-related diseases by administering a CTMP inhibitor are described herein. The CTMP inhibitor may be a CTMP gene silencing, a small molecule CTMP inhibitor, or a polynucleotide sequence encoding Zic2. Also provided are methods of preventing or reducing cellular senescence using CTMP inhibitors.

### CROSS-REFERENCE TO RELATED APPLICATION(S)

This application claims the benefit of and priority to U.S. Provisional Application No. 63/749,354 filed on Jan. 24, 2025, the content of which is incorporated by reference in its entirety.

### STATEMENT REGARDING FEDERALLY SPONSORED RESEARCH

This invention was made with government support under AG061047, AG061047-01A1S1 and NS099118 awarded by the National Institutes of Health. The government has certain rights in the invention.

### REFERENCE TO AN ELECTRONIC SEQUENCE LISTING

The content of the electronic sequence listing (18250600066.xml; Size: 14,975 bytes; and Date of Creation: Nov. 21, 2025) is herein incorporated by reference in its entirety.

### BACKGROUND

Cellular senescence is a process that imposes permanent proliferative arrest on cells in response to various stressors and has emerged as a potentially important contributor to aging and age-related diseases. The age-associated accumulation of senescent cells may be due to a combination of factors that include inefficient senescent cell removal by the immune system in old age and the resistance of senescent cells to apoptosis. Once a cell enters a state of growth arrest or senescence, it can display several hallmark cellular and molecular features that when detected (in combinations) can be used as biomarkers of cellular senescence.

It has been established that multiple diseases are caused by cellular senescence including cardiovascular diseases, metabolic diseases, musculoskeletal diseases, liver disease, kidney disease, and lung disease as well as neurodegenerative diseases such as Alzheimer's and Parkinson's. Therefore, strategies for reducing cellular senescence are of interest.

### SUMMARY

In an aspect, provided herein is a method of preventing or treating a disease associated with cellular senescence in a subject in need thereof, the method comprising: administering to the subject an effective amount of a CTMP inhibitor.

The CTMP inhibitor may comprise at least one of a polynucleotide sequence encoding Zic2, a CTMP gene silencing agent, or a small molecule compound that inhibits CTMP. The CTMP inhibitor may reduce at least one of CTMP expression and CTMP activity.

The disease associated with cellular senescence may be selected from a cardiovascular disease, a metabolic disease, a musculoskeletal disease, a liver disease, a kidney disease, a lung disease, and a neurodegenerative disease. In embodiments, the disease is a neurodegenerative disease. In embodiments, the disease is Alzheimer's disease. The subject may be a human.

In another aspect, provided herein is a method for preventing or reducing senescence in a cell, the method comprising contacting the cell with a CTMP inhibitor. The CTMP inhibitor comprises at least one of a polynucleotide sequence encoding Zic2, a CTMP gene silencing agent, or a small molecule compound that inhibits CTMP.

The polynucleotide encoding Zic2 may be packaged in a vector. The vector may be an M02 vector. The cell may be an iPSC. The cell may be derived from a subject having an age-related disease. The age-related disease may be Alzheimer's disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D. Increase of CTMP expression in rat fibroblast cultures stably transfected with ctmp cDNA. Rat fibroblast cells were transfected with M56-Vector or CTMP-ORF containing CTMP and mCherry expression codes. (A) mCherry expression in transfected cells. Scale bar: 50 μm. (B) Percentage of the number of mCherry positive cells in the total number of cells. Results are median with 95% CI (n=6) with the presence of values of individual sample. (C) Representative images of Western blotting. (D) Quantitative data of CTMP abundance. Results are median with 95% CI (n=10 to 19) with the presence of values of individual sample. Experiments were conducted for at least three times independently with 2 to 3 sets of samples each time. Normality was tested by Shapiro-Wilk test. Statistical analysis was done by one-way ANOVA.

FIGS. 2A-2C. Increase in SA-β-Gal positive and Ki67 negative cells with CTMP overexpression. (A) Representative images of Ki67 and SA-β-Gal staining of various groups. Scale bar: 25 μm. (B) Quantitative data of SA-β-Gal positive cells [SA-β-Gal (+) cells]. Results are median with 95% CI (n=6) with individual sample value. (C) Percentage of the number of cells positive for SA-β-Gal and negative for Ki67 [Ki67 (−) SA-β-Gal (+) cells] in total number of SA-β-Gal (+) cells. Results are median with 95% CI (n=6) with individual sample value. Experiments were conducted for three times independently with 2 sets of samples each time. Normality was tested by Shapiro-Wilk test. Statistical analysis was done by one-way ANOVA.

FIGS. 3A-3C. Decrease in colony formation and proliferation in rat fibroblast cells with CTMP overexpression. (A) Representative images of cell colonies stained with 1% crystal violet. (B) Quantitative data of cell colonies of various groups. (C) Quantitative data of cell counting kit-8 (CCK8) tests of various groups. Results are mean±S.D. (n=20) with the presence of values of individual sample. Experiments were conducted for four times independently with 4-6 sets of samples each time. Normality was tested by Shapiro-Wilk test. Statistical analysis was done by one-way ANOVA.

FIGS. 4A-4D. Increase in the number of cells with polyploid DNA in cells with CTMP overexpression. (A-C) Representative images of flow cytometry analysis of various groups. (D) Percentage of the number of cells with polyploid DNA in the total number of cells analyzed. Results are median with 95% CI (n=3) with the presence of values of individual sample. Experiments were conducted for three times independently. 25000 live events were collected for analysis each time. Normality was tested by Shapiro-Wilk test. Statistical analysis was done by one-way ANOVA.

FIGS. 5A-5D. Expression of CTMP and Zic2 changes with aging in the cerebral cortex. (A-B) Representative images of Western blotting to analyze the expression of CTMP and Zic2 in the prefrontal cortex of 2-month and 18-month old rats. (C) Quantitative data of CTMP abundance. (D) Quantitative data of Zic2. Results are mean=S.D. (n=8-10 rats per group) with the presence of values of individual animal. Statistical analysis was done by t-test. Data passed Shapiro-Wilk normality test. 2M: 2-month old, 18M: 18-month old.

FIGS. 6A-6C. Immunofluorescence staining of CTMP in the cerebral cortex of 18-month old rats and Zic2 in the cerebral cortex of 2-month old rats. (A) Co-staining of Zic2 or CTMP with NeuN. (B) Co-staining of Zic2 or CTMP with Iba1. (C) Co-staining of Zic2 or CTMP with s100β. Scale bar=50 μm.

FIG. 7. Co-staining of CTMP with MAP2 in human cerebral cortex. Scale bar=200 μm.

FIGS. 8A-8D. Direct regulation of CTMP expression by Zic2. (A) Chromatin immunoprecipitation results prepared using a rabbit anti-Zic2 antibody to precipitate the DNA samples of the cerebral cortex. The precipitants were amplified by PCR with a pair of primers. Normal rabbit IgG and an anti-RNA polymerase II were used as negative and positive controls, respectively. (B) Diagram of the constructs of the plasmid containing the presumptive CTMP promoter regions and coding sequence for luciferase. (C) Diagram of CTMP promoter activity assays. (D) Quantitative results of CTMP promoter activity. The luciferase (Luc) results were normalized by the corresponding Renilla luciferase (RL) data. Results are mean±S.D. (n=7-8) with the presence of values of individual sample. Experiments were conducted three times independently with 2 to 3 sets of samples each time. Data passed Shapiro-Wilk normality test. Statistical analysis was done by one-way ANOVA.

FIGS. 9A-9C. Reduction of CTMP expression in cells with overexpression of Zic2. (A) Representative images of Western blotting of various groups. (B) Quantitative data of Zic2 abundance. Results are median with 95% CI with the presence of values of individual sample (n=9-10). (C) Quantitative data of CTMP abundance. Results are mean±S.D. (n=7-9) with the presence of values of individual sample. Experiments were conducted three times independently with 2 to 3 sets of samples each time. Normality was tested by Shapiro-Wilk test. Statistical analysis was done by one-way ANOVA.

FIGS. 10A-10B. CTMP over-expression in iPSCs. iPSCs were transduced with lentivirus with or without CTMP-ORF and the stably transduced cells were selected out for the experiments. A: representative Western blotting images. B: quantitative results in mean±SD (n=9-12). Control: iPSCs without APPE693G mutation. AD: iPSCs with APPE693G mutation, B: blank group in which cells were not transduced with lentivirus, V: vector group in which cells were transduced with negative lentivirus vector particles, CTMP: CTMP group in which cells were transduced with lentivirus carrying CTMP-ORF. * P<0.05 for the comparison, ** P<0.01 for the comparison.

FIGS. 11A-11C. Evidence of successful transduction by lentivirus. iPSCs were transduced with lentivirus with or without CTMP-ORF and their stably transfected cells were selected out for the experiments. The lentivirus carried code of mCherry reporter. A: representative immunofluorescence images showing mCherry expression, scale bar=100 μm. B: quantitative results of cells expressing mCherry in median with 95% CI (n=4). C: representative images of flow cytometry showing mCherry expression. Control: iPSCs without APPE693G mutation. AD: iPSCs with APPE693G mutation, B: blank group in which cells were not transduced with lentivirus, V: vector group in which cells were transduced with negative lentivirus vector particles, CTMP: CTMP group in which cells were transduced with lentivirus carrying CTMP-ORF. P<0.0001 for the comparison.

FIGS. 12A-12C. CTMP over-expression decreased colony forming ability and cell proliferation. iPSCs were transduced with lentivirus with or without CTMP-ORF and their stably transduced cells were selected out for the experiments. A: representative images of cell colonies stained with 1% crystal violet. B: quantitative results of colony forming in median with 95% CI (n=6). C: cell counting results and results are median with 95% CI (n=26-27). Control: iPSCs without APPE693G mutation. AD: iPSCs with APPE693G mutation, B: blank group in which cells were not transduced with lentivirus, V: vector group in which cells were transduced with negative lentivirus vector particles, CTMP: CTMP group in which cells were transduced with lentivirus carrying CTMP-ORF. * P<0.05 for the comparison, ** P<0.01 for the comparison.

FIGS. 13A-13C. CTMP over-expression increased polyploid DNA and P16. iPSCs were transduced with lentivirus with or without CTMP-ORF and their stably transduced cells were selected out for the experiments. A: representative flow cytometry data showing polyploid DNA existence. B: quantitative results of polypoid DNA in mean±SD (n=8-12). C: results of P16 expression in median with 95% CI (n=10-22). Control: iPSCs without APPE693G mutation. AD: iPSCs with APPE693G mutation, B: blank group in which cells were not transduced with lentivirus, V: vector group in which cells were transduced with negative lentivirus vector particles, CTMP: CTMP group in which cells were transduced with lentivirus carrying CTMP-ORF. * P<0.05 for the comparison, ** P<0.01 for the comparison, **** P<0.0001 for the comparison.

FIGS. 14A-14F. CTMP over-expression inhibited autophagy in the AD iPSCs. iPSCs were transduced with lentivirus with or without CTMP-ORF and their stably transduced cells were selected out for the experiments. A: representative Western blotting images of LC3 I and LC3 II. B: quantitative results of the ratio of LC3 II and LC3 I in median with 95% CI (n=8-10). C: representative Western blotting images of P62. D: quantitative results of P62 in median with 95% CI (n=18-21). E: representative Western blotting images of beclin-1. F: quantitative results of beclin-1 in median with 95% CI (n=14-21). Control: iPSCs without APPE693G mutation. AD: iPSCs with APPE693G mutation, B: blank group in which cells were not transduced with lentivirus, V: vector group in which cells were transduced with negative lentivirus vector particles, CTMP: CTMP group in which cells were transduced with lentivirus carrying CTMP-ORF. * P<0.05 for the comparison, ** P<0.01 for the comparison.

FIGS. 15A-15B. AD iPSCs had increased PINK1. iPSCs were transduced with lentivirus with or without CTMP-ORF and their stably transduced cells were selected out for the experiments. A: representative Western blotting images showing PINK1 expression. B: quantitative results of PINK1 in median with 95% CI (n=8-15). Control: iPSCs without APPE693G mutation. AD: iPSCs with APPE693G mutation, B: blank group in which cells were not transduced with lentivirus, V: vector group in which cells were transduced with negative lentivirus vector particles, CTMP: CTMP group in which cells were transduced with lentivirus carrying CTMP-ORF. * P<0.05 for the comparison.

### DETAILED DESCRIPTION

As illustrated in the Examples, the inventors found that carboxyl terminal modulator protein (CTMP) increases with aging in the brain, and that overexpression of CTMP induces cellular senescence. The inventors also found that Zic2 decreases with aging in the brain and directly inhibits the expression of CTMP. Additionally, the inventors found that CTMP induces cell senescence in both control and AD iPSCs and reduces autophagy in Alzheimer's disease (AD) iPSCs, which have unhealthy mitochondria.

In a first aspect, provided herein is a method for treating a senescence-associated disease in a subject in need thereof by administering an effective amount of a CTMP inhibitor. The CTMP inhibitor may reduce the expression or activity of CTMP.

Diseases associated with cellular senescence include, but are not limited to, cardiovascular diseases, metabolic diseases, musculoskeletal diseases, liver diseases, kidney diseases, lung diseases, and neurodegenerative diseases. Cellular senescence is linked to several age-related chronic diseases, such as Alzheimer's, Parkinson's, type 2 diabetes, cardiovascular disease, and osteoarthritis. In embodiments, the subject has a neurodegenerative disease. In embodiments, the subject has Alzheimer's disease.

Cellular senescence is the cessation of cell division. Cells may undergo senescence in response to various stressors such as DNA damage, oncogene activation, or telomere shortening.

Carboxyl terminal modulator protein (CTMP) (NCBI Gene ID: 117145) is a mitochondrial protein that acts as a negative regulator of the of Akt (Protein Kinase B) signaling pathway, and is involved in cell survival and growth. CTMP is released from the mitochondria into the cytosol upon apoptotic stimuli, sensitizing cells to apoptosis, and plays a role in various diseases like cancer, brain injury, and muscle atrophy.

The CTMP inhibitor may comprise at least one of a polynucleotide sequence encoding Zic2, a CTMP gene silencing agent, or a small molecule compound that inhibits CTMP. The CTMP inhibitor may also comprise an agent that increases the activity or expression of Zic2.

Zic2 (NCBI Gene ID: 7546) is a zinc finger protein that acts as a transcription factor, and regulates expression of various genes. In embodiments, the polynucleotide sequence encoding Zic2 comprises or consists of SEQ ID NO: 7 or a sequence having at least 90% identity thereto. In embodiments the polynucleotide comprises or consists of a sequence that encodes human Zic2 protein, wherein the sequence comprises or consists of mlldagpqfpaigvgsfarhhhhsaaaaaaaaaemqdrelslaaaqngfvdsaaahmgafklnpgahelspgqssaftsqgpgayp gsaaaaaaaaalgphaahvgsysgppfnstrdflfrsrgfgdsapgggqhglfgpgagglhhahsdaqghllfpglpeqhgphgsqnv Ingqmrlglpgevfgrseqyrqvasprtdpysaaqlhnqygpmnmnmgmnmaaaaahhhhhhhhhpgaffrymrqqcikqel ickwidpeqlsnpkkscnktfstmhelvthvsvehvggpeqsnhvcfweecpregkpfkakyklvnhirvhtgekpfpcpfpgcgk vfarsenlkihkrthtgekpfqcefegcdrrfanssdrkkhmhvhtsdkpylckmcdksythpsslrkhmkvhesspqgsesspaas sgyesstppglvspsaepqsssnlspaaaaaaaaaaaaaaavsavhrgggsgsggagggsgggsgsggggggaggggggssgggsg tagghsglssnfnewyv (SEQ ID NO: 9) or a sequence having at least 90% identity thereto.

A gene silencing agent may reduce expression of a target gene or a molecule that positively regulates said target gene. The gene silencing agent may comprise a nucleic acid sequence that is capable of inducing RNA interference (RNAi). The term “RNA interference” refers to a process in which RNA molecules inhibit gene expression or translation by neutralizing targeted mRNA molecules. To achieve an RNAi effect, for example, RNA having a double strand structure containing the same base sequence as that of the target mRNA may be used. Gene silencing agents include, but are not limited to, small interfering RNA (siRNA), short hairpin RNA (shRNA) microRNA (miRNA), miRNA zippers, and antisense oligonucleotides (ASOs). Any RNA molecule that is capable of silencing gene expression of a target gene may be used in connection with the present disclosure.

The polynucleotide encoding Zic2 and the gene silencing agent may be packaged in delivery vehicles. The term “delivery vehicle”, as used herein, refers to a molecular compound suitable for delivering the gene silencing agent to a cell. Delivery vehicles include, but are not limited to, vectors, lipid nanoparticles, and polymeric nanoparticles. In embodiments, the polynucleotide encoding Zic2 is in a construct and operably linked to a promoter. In embodiments, the gene silencing agent is in a construct and operably linked to a promoter. The term “construct” refers to a recombinant polynucleotide, i.e., a polynucleotide that was formed artificially by combining at least two polynucleotide components from different sources (natural or synthetic). For example, the construct may comprise promoters and elements that are (1) associated with another gene found within the same genome, (2) from the genome of a different species, or (3) synthetic. As used herein, the term “promoter” refers to a DNA sequence that regulates the transcription of a polynucleotide. Typically, a promoter is a regulatory region that is capable of binding RNA polymerase and initiating transcription of a downstream sequence. Promoters may be derived in their entirety from a native gene, may be composed of elements derived from multiple regulatory sequences found in nature, or may comprise synthetic DNA segments. A promoter is “operably linked” to a polynucleotide if the promoter is connected to the polynucleotide such that it may affect transcription of the polynucleotide. Constructs can be generated using conventional recombinant DNA methods. In embodiments, the construct is packaged in a vector. As used herein, the term “vector” refers to a virus particle that is used to deliver genetic material into cells. Vectors include, without limitation, a yeast artificial chromosome, bacterial plasmid (e.g., naked or contained in liposomes), phagemid, shuttle vector, cosmid, virus (e.g., Sendai viruses, lentiviruses, retroviruses, adenoviruses, and adeno-associated viruses), chromosome, mitochondrial DNA, plasmid DNA, and nucleic acid fragment. In exemplary embodiments, the polynucleotide encoding Zic2 is packaged in a lentiviral vector. In embodiments, the polynucleotide encoding Zic2 is in an M02 vector.

The term “nanocarrier,” “nanoparticle,” or “nanostructure” refers to a nanomaterial used as a transport module for another substance. Nanocarriers include, without limitation, polymeric nanoparticles, carbon-based materials, dendrimers, and lipid-based carriers, such as liposomes. For example, the gene silencing agent may be an siRNA in a lipid nanocarrier.

As used herein, the terms “small molecular therapeutic agent”, “small molecule compound”, and “small molecule drug” refer to a chemical compound or pharmaceutically acceptable salt thereof having a therapeutic effect and/or enhancing the therapeutic effect of an immunotherapy. Small-molecule drugs are typically comprised of 20 to 100 atoms and have a molecular mass of less than 1000 g/mol or 1 kilodalton [kDa]. Small-molecules drugs can typically be administered by a variety of routes (including orally) and can pass through cell membranes to reach intercellular targets.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, “preventative,” “preventing,” “prevent” and the like refer to partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms.

As used herein, the term “administering”, refers to dispensing, delivering, or applying the therapeutic agent (e.g. CTMP inhibitor, Zic2 activator), to a subject by any suitable route for delivery of the substance to the desired location in the subject, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route. The preferred route may vary with, for example, the subject's pathological condition or age or the subject's response to therapy or that is appropriate to the circumstances.

Suitable regimes for initial administration and further doses or for sequential administrations also are variable, may include an initial administration followed by subsequent administrations, but nonetheless, may be ascertained by the skilled artisan from this disclosure, the documents cited herein, and the knowledge in the art.

The terms “subject” and “patient” are used herein interchangeably to refer to a mammal to be treated by the methods and compositions described herein. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. In embodiments, the subject is a human.

The terms “effective amount” or “therapeutically effective amount” refer to an amount sufficient to effect beneficial or desirable biological and/or clinical results. The amount of the pharmaceutical composition that is therapeutically effective may vary depending on the particular pathogen or the condition of the subject. Appropriate dosages may be determined, for example, by extrapolation from cell culture assays, animal studies, or human clinical trials taking into account body weight of the patient, absorption rate, half-life, disease severity and the like. The dosage lies preferably within a range of circulating concentrations that include the ED50 with little or no toxicity. The dosage may vary within this range depending upon the dosage form employed and the route of administration utilized.

The CTMP inhibitor may be formulated in pharmaceutical composition comprising the inhibitor and a pharmaceutically acceptable carrier.

The term “pharmaceutically acceptable carrier,” as used herein, means a non-toxic, inert solid, semi-solid or liquid filler, diluent, encapsulating material or formulation auxiliary of any type. Suitable pharmaceutically acceptable carriers include, but are not limited to, diluents, preservatives, solubilizers, emulsifiers, liposomes, nanoparticles and adjuvants.

Pharmaceutically acceptable carriers are well known to those skilled in the art and include, but are not limited to, 0.01 to 0.1 M and preferably 0.05M phosphate buffer or 0.9% saline. Additionally, such pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The composition may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final composition. The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity, such as sodium chloride or dextrose. The pH can be adjusted with acids or bases, such as hydrochloric acid or sodium hydroxide. The preparation can be enclosed in ampoules, disposable syringes or multiple-dose vials made of glass or plastic. For convenience of the patient or treating physician, the dosing formulation can be provided in a kit containing all necessary equipment (e.g., vials of drug, vials of diluent, syringes and needles) for a course of treatment (e.g., 7 days of treatment).

Sterile injectable solutions can be prepared by incorporating the active chemical compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Capsules are prepared by mixing the chemical compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders. Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The chemical compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects. A lubricant can be used in a tablet formulation to prevent the tablet and punches from sticking in the die. The lubricant can be chosen from such slippery solids as talc, magnesium and calcium stearate, stearic acid, and hydrogenated vegetable oils. Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

In embodiments, the method comprises administering any additional agent or therapy that treats the senescence-associated disease. The small molecule compound or gene silencing agent that inhibit CTMP, the polynucleotide encoding Zic2 or Zic activator, and/or the additional agent or therapy may be administered in any order. The formulations can also be administered by two or more routes, where the delivery methods are essentially simultaneous, or they may be essentially sequential with little or no temporal overlap in the times at which the composition is administered to the subject.

In a second aspect, provided herein is a method for preventing or reducing senescence in a cell, the method comprising contacting the cell with a CTMP inhibitor.

“Contacting” a cell or a cell culture refers to adding the agent or composition to a medium or buffer comprising the cell culture. Contacting the cell may comprise transducing or transfecting the cell. The term “transduced” or “transfected” refers to the ability of an exogenous polynucleotide to be introduced to a cell, particularly introduced to the nucleus of a cell. The cell may be derived from a subject having an age-related disease or a disease associated with cellular senescence.

In embodiments, the cell is an induced pluripotent stem cell (iPSC). An iPSC is a type of pluripotent stem cell that can be generated directly from a somatic cell. A somatic cell is a biological cell that is not a gamete, germ cell, gametocyte, or undifferentiated stem cell.

In a third aspect, provided herein is a method for inducing autophagy in a cell, the method comprising contacting the cell with a polynucleotide sequence encoding CTMP. In embodiments, the polynucleotide sequence encoding CTMP comprises SEQ ID NO: 8 or a sequence having at least 90% identity thereto. In embodiments the polynucleotide comprises or consists of a sequence that encodes human CTMP protein, wherein the sequence comprises or consists of

(SEQ ID NO: 10)
MLRSCAARLRTLGALCRPPVGRRLPGSEPRPELRSFSSEEVILKDCSVPNPSWNKDLRLL
FDQFMKKCEDGSWKRLPSYKRTPTEWIQDFKTHFLAWLMAGRCGSRL

or a sequence having at least 90% identity thereto.

The terms “polynucleotide,” “polynucleotide sequence,” “nucleic acid,” and “nucleic acid sequence” refer to a nucleotide, oligonucleotide, polynucleotide (which terms may be used interchangeably), or any fragment thereof. A “polynucleotide” may refer to a polydeoxyribonucleotide (containing 2-deoxy-D-ribose), a polyribonucleotide (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs. These phrases also refer to DNA or RNA of genomic, natural, or synthetic origin (which may be single-stranded or double-stranded and may represent the sense or the antisense strand).

As used herein, the term “nucleic acid” or “polynucleotide” refers to deoxyribonucleic acid (DNA), ribonucleic acid (RNA) and DNA/RNA hybrids. Polynucleotides may be single-stranded or double-stranded. Nucleic acids include, but are not limited to: pre-messenger RNA (pre-mRNA), messenger RNA (mRNA), RNA, short interfering RNA (siRNA), short hairpin RNA (shRNA), microRNA (miRNA), ribozymes, synthetic RNA, genomic RNA (geRNA), guide RNA, tracRNA, crRNA, sgRNA, plus strand RNA (RNA (+)), minus strand RNA (RNA (−)), synthetic RNA, genomic DNA (gDNA), PCR amplified DNA, complementary DNA (cDNA), synthetic DNA, or recombinant DNA.

The terms “oligonucleotide”, “polynucleotide”, and “nucleic acid” are used to refer to DNA or RNA molecules, or fragments thereof. These terms include double- and single-stranded DNA, as well as double- and single-stranded RNA. A “polynucleotide” or “oligonucleotide” may refer to a polydeoxyribonucleotide (containing 2-deoxy-D-ribose), a polyribonucleotide (containing D-ribose), and to any other type of polynucleotide that is an N glycoside of a purine or pyrimidine base. Oligonucleotides are typically shorter in length (e.g. 2-100 nucleotides), while polynucleotides are longer chains of nucleotides (e.g. longer than 100 nucleotides). For use in the present compositions and methods, an oligonucleotide also can comprise nucleotide analogs in which the base, sugar, or phosphate backbone is modified as well as non-purine or non-pyrimidine nucleotide analogs. These phrases also refer to DNA or RNA of genomic, natural, or synthetic origin (which may be single-stranded or double-stranded and may represent the sense or the antisense strand).

### MISCELLANEOUS

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The cited references are incorporated by reference herein in their entireties. In the event that there is an inconsistency between a definition of a term in the specification as compared to a definition of the term in a cited reference, the term should be interpreted based on the definition in the specification.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

### Introduction

Carboxyl terminal modulator protein (CTMP) is an endogenous inhibitor of Akt (Maira et al. 2001), an important signaling molecule that is involved in various functions, such as cell survival, tumor cell growth and inflammation (Manning and Toker 2017). Consistent with this pattern of molecular interaction, CTMP has been found to increase inflammation and reduce cell survival after detrimental stimuli including ischemia (Li, Shan, and Zuo 2018; Miyawaki et al. 2009; Wang et al. 2014; Yu, Deng, and Zuo 2014) and to decrease pressure-induced cardiac hypertrophy (X. Liu et al. 2018). CTMP may inhibit cancer cell growth (Knobbe et al. 2004), although an opposite finding has been reported (Y. P. Liu et al. 2013). Our recent study has shown that CTMP is increased with aging in the brain and that this increase may contribute to the decreased brain ischemic tolerance in old rats (Li, Shan, and Zuo 2018). These findings suggest that CTMP may be involved in cell senescence because cell senescence is presented with aging and is involved in various diseases, especially aging-related diseases (Kudlova, De Sanctis, and Hajduch 2022). To examine the role of CTMP in cell senescence, CTMP was overexpressed in rat fibroblasts to determine the senescence of these cells.

Although CTMP has been identified for more than 20 years (Maira et al. 2001), very little is known about how CTMP expression is regulated. We examined the sequence 2000 bp upstream of the initiation codon of rat ctmp gene and found Zic2 binding sites in the sequence. Zic2 is a member of zinc finger protein that regulates expression of various genes. One possible mechanism for this regulation is that Zic2 inhibits the transcriptional activity of β-catenin/transcription factor 4 complex (Pourebrahim et al. 2011).

Based on the information, we hypothesize that Zic2 regulates CTMP expression and that this regulation contributes to the increase of CTMP with aging. To test these hypotheses, we examined the expression of Zic2 with aging in the brain and performed biochemical analyses to determine the effects of Zic2 on CTMP expression.

### Materials and Methods

The animal protocol was approved by the Institutional Animal Care and Use Committee of the University of Virginia (Charlottesville, VA, USA; protocol number 3114). Fischer 344 male rats from the National Institutes of Health, USA, at the ages of 2 or 18 months were used to harvest brain tissues. These animals were maintained in standard living conditions and did not have a survival surgery or any treatments.

### Western Blotting Analyses

The cerebral cortices of male Fischer 344 rats or cell cultures were harvested and lysed with RIPA buffer (Thermo Fisher Scientific, Waltham, MA, USA). Twenty microgram protein was separated on 12% SDS-PAGE gel and then transferred to polyvinylidine difluoride membranes. Primary antibodies were anti-Zic2 antibody (1:1000 dilution, Abcam, Cambridge, UK), anti-CTMP antibody (1:1000 dilution, Cell signaling Technology, Danvers, MA, USA) and anti-β-actin antibody (1:1000 dilution, Cell Signaling Technology). Secondary antibody was mouse anti-rabbit antibody conjugated with horseradish peroxidase (1:5000 dilution, Santa Cruz Biotechnology, Dallas, TX, USA). The results of bands corresponding to Zic2 and CTMP proteins were normalized to the values of corresponding internal control proteins (Li, Shan, and Zuo 2018). At least 7 animals or samples of cell cultures in each group were analyzed.

Generation of Rat Fibroblasts with Stable Overexpression of CTMP

To determine the role of CTMP in cell senescence, cells stably over-expressing CTMP were needed for the studies. It was decided to use rat primary fibroblasts because fibroblasts but not major brain cells, such as neurons, astrocytes or microglia, could be used to generate a cell line to stably over-express a target protein. Rat primary fibroblasts were isolated from 8-week-old Fischer 344 rat lungs and cultured in Eagle's minimum essential medium with 15% FBS, 1× penicillin/streptomycin, non-essential amino acids and sodium pyruvate. Primary rat fibroblasts were seeded on 6-well plate and transfected at 80% confluence with a plasmid containing CTMP open reading frame and mCherry code (CTMP-ORF, NM001025017.1, Genecopoeia) or M56-Vector control (M56-Vector, Genecopoeia) by using EndoFectin Max transfection reagent (Genecopoeia).

NM001025017.1-
(SEQ ID NO: 8)
gtccggaatccgcgtgccgggtagtgtggctctttctggttgagaggacattgcagcgtcctgccctcggcttgaacgatgttgagaagctgcgct
atgcgcctgcgcactcttggggccacgcctgcaaggcggcctgaagcaacgaggaggttattttcttctgaggaagtcgtctgtaaggactatgct
ctccctaaccccagctggactaaggacctaagactcctttttgaccagttcatgaagaaatgtgaagatggctcctggaaacgtttgccttcatac
agacaaaaccctcctcaagcccttcaagagttccaaacccactttgttgactcaaagtttaagaaagaagaacaaatgtcaaaggcacaaca
gttcaccagaagcttagaagaaggactaggctttgagtacgcgatgttctataataaggctgagaagagaattgtctgcttgtttcaaggaggt
cttcacctgcaaggaatgcctggatttgttcatggaggtgccattgcaaccatcatcgacatcactgccggcatgtgtgcattttctgagggca
ttgtcatgactgccaatctcaacatcgactataaaaaacctatcccccttctttctgttgttgtggtaaatagccaacttcaaaaaattgaaggaa
ggaaactttttgtttcctgtactattcaaagtactgatgagaagactttacatacacaggcaacagccttatttataaagctggatcctgacaaac
ctttgacataaaagaactctggtgagttccacaccgttctgcatgaggtccccgccccctccagaagtagcacctatctccactcctgtcaca
gctgctgaatcccccagggaaagtctgctctcacagctccttcagcgcagcctctgacgtggctgcaggaactcactctcaccttccagagtt
atcataagaagtactgcccgggggcgtctcagcagtaatctgtatgtccacgtgacaactgtgtggttctaactcgtgaacagacttcaaagc
taacaagcataggacctgctctgctgtgggctgcagaaaaaaacaaaagaccatattgagacttgggctgcaaatgtggaaatgcacgaaa
accacaggcagggatagatccacagagcgttgagtgacgtgcgtgtgtgcacaagccgcgccgggtcttacagaactaagtttgttgtatct
gaacaaactctctttggccacatcaggaacacagctcattgctgtgggaatggcccccacacatcattgtctggggccctagagattaaaata
ctgttcatttttctggcctgtgctgctgtagcttagaacactgtagtctgtcacccctggaagaaggctttgttacatttctgggactgtaatgaa
gagctacaagctgggtggtttaaacaaataaaaaacgtcactggcacagccccaggggccagcagtctaaaagtattaacaaaacgttgcttc
ttaaggttctggggggcactttcacatctctcctggcttctaccaaggaagcatccagctttccttggtttttagacacagcataattcctaattg
catctcttttatcatcttctctcggttgtatccacatttccctttgtgaatgtttatttatttatttgtctgggtgggtgtgtgtgttcaggtgct
aaaataaattgggagattagtgttctacaaagtgaaaaaaaaaaaaaaaaaaaaaaaaa.

Seventy-two hours later, 200 μg/ml geneticin (ThermoFisher) was added for two weeks for stable transfection selection. The overexpression of CTMP in these fibroblasts was confirmed by immunostaining and Western blotting. The percentage of the number of cells expressing transfected proteins in the total number of cells was quantified in the images taken by a confocal microscope Zeiss LSM700.

The cells transfected with CTMP-ORF or M56-Vector were expanded and kept in Eagle's minimum essential medium with 200 μg/ml geneticin for the following studies.

### Senescence-Associated Beta-Galactosidase (SA-β-Gal) and Antigen Kiel 67 (Ki67) Staining

Primary rat fibroblasts transfected with or without CTMP-ORF or M56-Vector were seeded on 8-well chamber slide (5000 cells/well). SA-β-Gal staining was performed when cells grew to ~50% confluence by using beta-gal staining kit (Abcam) to avoid confluence-induced SA-β-Gal activity increase (Yang and Hu 2005). Then, slides were stained with an anti-Ki67 antibody (1:200, Abcam) overnight and counterstained with Hoechst33342 (1:1000, ThermoFisher Scientific). Secondary antibody used was Alexa Fluor 647 donkey anti-rabbit IgG (H+L) antibody (1:200, Invitrogen). Images were taken by a Leica Thunder TIRF. Experiments were conducted three times independently with 2 slides for each experimental condition. Six images were taken from each slide at 20× objective lens. The percentage of the number of cells that were positive for beta-galactosidase staining and negative for Ki67 in the number of cells positive for beta-galactosidase staining in each slide was quantified (the average of the 6 images of the slide).

### Cell Proliferation and Viability Tests

As previously described (Lai et al. 2019), colony forming ability was assessed by plating 50 cells/well on 12-well plate for two weeks. Cells were fixed and stained with 1% crystal violet for 30 min. Colonies with more than 50 cells were counted. The percentage of the number of colonies in the total number of cells seeded was calculated to reflect the colony-formation efficiency.

Cell counting kit (Abcam) was used to determine the proliferation of cells. Briefly, 5000 cells/well were plated on 96-well plate for 48 hours and then incubated with 10 μl/well of WST-8 solution at 37° C. for 3 hours before measuring the absorbance at 460 nm.

### Flow Cytometry

As previously described (Xu, Feng, and Zuo 2008), cells were harvested and washed 2 times in PBS. Cells were re-suspended in 1.2 ml PBS (Ca2+ and Mg2+ free) and mixed with 3 ml ice cold 95% ethanol dropwise while being vortexed. Cells were fixed in the final 70% ethanol for at least 30 min. Cells were then washed twice with 12 ml PBS (centrifuged at 2000 rpm for 10 min) and then re-suspended in cell staining buffer (Biolegend, San Diego, CA, USA) containing 0.1 μg/ml 4′6-diamidino-2-phenylindole (DAPI) (Biolegend) to stain 1×106 cells on ice for 30 min. Cells were analyzed by a flow cytometer Attune NxT. The percentage of cells with polyploid DNA was measured.

### Immunofluorescence Staining

Immunofluorescence staining was performed as previously described (Shan et al. 2019; Zeng et al. 2021). Briefly, rats were perfused with normal saline under deep anesthesia. Brain slices from Bregma −2 to −5 mm were fixed in 4% paraformaldehyde for 48 h at 4° C. and then embedded in paraffin. Five-micrometer thick coronal sections were mounted on slides. After antigen retrieval in sodium citrate buffer (10 mM sodium citrate, 0.1% tween 20, pH 6.0) for 20 min, sections were blocked in 10% donkey serum plus 1% bovine serum albumin (BSA) in Tris-buffered saline (TBS) containing 0.1% triton-X 100 for 2 hours at room temperature and then incubated with the corresponding primary antibodies at 4° C. overnight. Primary antibodies were rabbit polyclonal anti-CTMP antibody (1:50 dilution, Abcam, Waltham, MA, USA), mouse monoclonal anti-neuronal nuclear protein (NeuN) (1:200 dilution, Millipore Sigma, Burlington, MA, USA), rabbit polyclonal anti-Zic2 antibody (1:100 dilution, Abcam), mouse monoclonal anti-S100ß (1:100 dilution, Santa Cruz Biotechnology) or goat polyclonal anti-ionized calcium binding adaptor molecule 1 (Iba1) (1:200 dilution, Abcam). On day 2, sections were rinsed with TBS containing 0.1% triton-X 100 and then incubated with donkey anti-mouse/goat/rabbit IgG antibody conjugated with Alexa Fluor 488 or Alexa Fluor 594 (1:200 dilution, Thermo Fisher Scientific) for 1 hour at room temperature. Slides were mounted for microscopic observation.

In another study to determine the human relevance of the findings from rats, human cerebral cortex sections were provided by the University of Virginia Biorepository and Tissue Research Facility, Charlottesville, VA. They were incubated with mouse monoclonal anti-MAP2 antibody (1:500 dilution, Invitrogen) or rabbit polyclonal anti-CTMP antibody (1:500 dilution, Abcam) and then with secondary antibody as described above for rat brain sections.

### Chromatin Immunoprecipitation Assay

The chromatin immunoprecipitation (ChIP) assay was conducted as we described before with some modifications (Feng et al. 2014). Briefly, cerebral cortex of 2-month old Fischer 344 male rats was cross-linked by 1.5% formaldehyde for 15 min at room temperature. The cross-linked tissue was then grinded and passed through a 28-gauge needle to prepare single cell suspension. Sonication was applied to fragment the DNA into 500-1000 base pairs in lysis buffer (1% SDS, 10 mM EDTA, 50 mM Tris-HCl, pH 8.1) containing protease inhibitors. The resulting DNA fragments were incubated with an anti-Zic2 antibody (2 μg/ml, Abcam) in ChIP dilution buffer overnight (0.01% SDS, 1.1% Triton X-100, 1.2 mM EDTA, 16.7 mM Tris-HCl, pH 8.1, 167 mM NaCl). Normal rabbit IgG (Millipore Sigma) and an anti-RNA polymerase II antibody (2 ug/ml, Millipore Sigma) were used as negative and positive control, respectively. On day 2, 20 μl Dynabead slurry (Thermo Fisher Scientific) per 1 ml sample was added and incubated at 4° C. for 2 hours on a rotator. After incubation, beads were pelleted with magnet and washed twice with following buffers, low salt immune complex wash buffer (0.1% SDS, 1% Triton X-100, 2 mM EDTA, 20 mM Tris-HCl, pH 8.1, 150 mM NaCl), LiCl immune complex wash buffer (0.25 M LiCl, 1% NP40, 1% deoxycholate, 1 mM EDTA, 10 mM Tris-HCl, pH 8.1) and 1× Tris-EDTA (TE) buffer (10 mM Tris-HCl, 1 mM EDTA, pH 8.0). After last wash, beads were re-suspended in elution buffer (1×TE, pH 8.0, 1% SDS, 150 mM NaCl, 5 mM DTT). Reverse cross-links were performed by incubating samples at 65° C. for 5 hours. After proteinase K treatment, phenol/chloroform extraction was performed to precipitate DNA. The final DNA sample was dissolved in 10 mM Tris-HCl, pH 8.1, for further use.

Primers used to amplify the ctmp fragment were as follows:

F:
(SEQ ID NO: 1)
5′-GGCTAACTCTATCCAATCCA-3′,

R: 5′-GTAGGTCACTCGCACATT-3′ (SEQ ID NO: 2), PCR product size would be 201 bp.

Primers for the positive control glyceraldehyde-3-phosphate dehydrogenase (gapdh) were:

F:
(SEQ ID NO: 3)
5′-TCACTACCGAAGAACAACGA-3′,

R: 5′-GGATAGGACTCAGGGAATACA-3′ (SEQ ID NO: 4), PCR product size would be 248 bp.

### Luciferase Assay

To determine the regulation of the transcription factor Zic2 on ctmp gene, plasmid containing Zic2 open reading frame cDNA (rat) was purchased from Genecopoeia (Rockville, Maryland, USA). A presumptive promoter sequence of the rat ctmp gene in chromosome 2 from −1176 bp to 174 bp was amplified and engineered into the pGL3-enhancer vector (pGL3-Ctmp-Luc+-enhancer, Promega, Madison, WI, USA) (Feng et al. 2014). Constructed plasmid was sequenced to confirm the accuracy of DNA before use in experiment. Primers used to amplify the sequence containing ctmp promoter region were

ctmp-F:
(SEQ ID NO: 5)
5′-tccGGTACCATGTTCCAGATTAGCAACCT-3′
and
ctmp-R:
(SEQ ID NO: 6)
5′-ggcAAGCTTCAATGTCCTCTCAACCAGAA-3′.

KpnI and HindIII enzymatic sites are labeled in bold.

293 FT cells were cultured in 12-well plates in Eagle's Minimum Essential Medium containing 10% fetal bovin serum. When cells grew to 50% confluence, Zic2-ORF plasmid, pGL3-Ctmp-Luc+-enhancer and pRL-TK (Promega) were co-transfected into cells with Lipofectamine LTX (Thermo Fisher Scientific). For control group, backbone of the plasmid for containing the Zic2 open reading frame, pReceiver-M02 plasmid, was used for co-transfection together with pGL3-Ctmp-enhancer and pRL-TK. The Renilla luciferase vector pRL-TK was cotransfected as internal control. Cells were lysed for luciferase activity test 48 h after transfection. Luciferase activity was measured by a kit (Dual-Luciferase Reporter Assay System, Promega). Three independent experiments with 2 to 3 sets of samples each time were conducted.

### Over-Expression of Zic2

To facilitate the transfection with plasmid and the expression of target proteins, 293 FT cells were used in the over-expression experiments. They were transfected with a plasmid containing rat Zic2 open reading frame cDNA (Zic2-ORF, NM_001108392, Genecopoeia, Rockville, MD, USA) or pReceiver-M02 plasmid (vector control, Genecopoeia) for 48 hours.

NM_001108392-
(SEQ ID NO: 7)
cgcgcctcctccgccgccgcctcctcttcctctccgcgccttcgctcggcgcccggccgcctgaggcagatacaggcggcggcggccgg
cgcagtagcccggctcccgaggctgcagtcgccagtgtttccaccgccggttcgcggagcccggcgtccagaggacgcgcctcgccga
tcaggaggggaaatccaggcagcagggctcaccggggccggcggggcggccgggcgcgctggccatgcttctggacgcggggccg
cagttcccggccatcgggggggcagcttcgcgcgccaccaccaccactcggccgcggcagcggcggctgcggcggccgagatgcag
gaccgggagctgagcctggcggcggcacagaacggcttcgtggactcggccgcagcgcacatgggcgccttcaagctcaaccccggg
gcacacgaactgtctcctggtcagagttcggcgttcacgtcgcaaggtccgggcgcctacccgggttcggctgcagcggccgctgcagcc
gcggctctagggccccacgccgctcacgttggctcttattcggggcctccctttaattccacccgggacttcctgttccgtagccggggcttc
ggggactcggcgccgggaggcggccagcacgggctcttcggacccggcgccggcggcctccaccatgcgcactcggacgcgcagg
gccaccttctcttccctggcctcccggaacagcacgggccgcacgcctcgcagaacgtgctcaatgggcaaatgcgcctagggctgccg
ggcgaagtgttcgggcgctcagagcaataccgccaagtggccagcccgcggaccgaccettactcggcggcgcagctccacaaccagt
acggccctatgaatatgaacatggggatgaacatggcagcggccgcagcccaccaccaccaccaccatcaccaccctggtgcctttttcc
gctacatgcggcagcagtgcatcaagcaagagctcatctgcaagtggatcgacccggagcagctaagcaatcccaagaaaagctgcaac
aaaactttcagcaccatgcacgagctggtgacccacgtctctgtagagcatgtcggcggcccggaacagagcaaccacgtctgcttctggg
aggagtgtccacgcgagggcaaaccctttaaggccaaatacaaactggtcaaccacatccgcgtgcacacgggcgagaaacccttccctt
gtcctttcccgggttgtggcaaggtcttcgcacgctccgagaacctcaagatccacaaaagaactcacacaggggagaaacctttccagtgt
gagttcgagggctgtgaccgacgcttcgccaacagcagcgacaggaagaagcacatgcacgtccacacctcagataagccctatctctgc
aagatgtgcgacaagtcctacacgcatcccagctccttgeggaagcacatgaaggtccatgagtcctcccctcagggctccgagtcctccc
cggctgccagctctggctacgagtcgtccacacccccagggttggtgtcccccagcgcagagccacaaagcagctccaacctgtcccca
gcagcggcagcggcggcggcagcggctgcagcagcggcggccgcggtgtccgcagtgcaccgaggcgcgggttctggcagtagcg
gctccggagggggctcggcggccggcagcggtgggggcggcggcggggcgggcggcgggggcggcggcagctctggcgggggc
agcgggacagccggaggccatagcggcctctcctccaacttcaatgaatggtacgtgtgaagggccaggcctttctcccattccctgttccc
ttatccaccgtcgccctcccaaaaacccaccgagggcaccttaggatcatcttactaaaattatgagtctgatttttatggtgaaaaaaattttac
cagcagaaggatttttaaaagtttttttttctttttaaagaaaacaatctaggcatgaaaagcaacatctctctgtatctctgaagctgaaaacat
aaaaaaaattttttgaagaaagtaaactccacaaaaatatcgagccaaacttccctagcaacccccagcccactttccttcccatatgcttcccag
tctttttgacaaactgtacatagcggactccttccttttccacggtgattttaatggcttattggtggatagaagtttgtccatttgtaaactccg
gattgcgttccctcctgcctttctcccattccccttccccaagtgacggaccttttttttttccttttagtttacccagtctttttttttttaaag
taatgtggaagaaaatggtttattttgtattgtggtattaaatattgtgttccttttaaatgaggcaacttgattgtaaacttcatgcgactatag
actggaaaatatgagccgtgccaaagtttcccttctgtttcttcaacacaccaacccatagcacacatcaccaccaccaccaacgcttgtgaatgt
atttttctgttagctgggtttacatgtgatgtttcagtgcttttgcgagttcgatttgttagttcttgtatgaaagtttgggggtggggtgggtgg
gagtaaacgttgtgccgttagctttttccgaaataacacccttctgtaaatatccgttgccatatttatccatttgtaattaaattatggtattaa
cttgctacagaggaaacaatatttataaagaatgtttcttaactataaatatgtacaattgtgggcataaactgtttcagatttttttatttgaag
gtttaagtggtttgatcatttcttgtgatgttttgagagtaatgcatacagaaatataataaaatgtgttgaaactgca

Cells were lysed with RIPA buffer (ThermoFisher Scientific). Twenty microgram protein was separated on 10% SDS-PAGE gel and then transferred to polyvinylidine difluoride membranes. Primary antibodies were anti-Zic2 antibody (1:1000 dilution, Abcam, UK), anti-CTMP antibody (1:1000 dilution, Cell Signaling Technology, Danvers, MA, USA) and anti-β-actin antibody (1:1000 dilution, Cell Signaling Technology). Secondary antibody was goat anti-rabbit antibody conjugated with horseradish peroxidase (1:5000 dilution, Santa Cruz Biotechnology). The results of bands corresponding to Zic2 and CTMP proteins were normalized to the values of β-actin.

### Statistical Analysis

Results are presented as mean±S.D. (parametric data in normal distribution) or median with 95% CI (non-parametric data or parametric data in non-normal distribution). The data of individual animal or experiment are presented in the figures. Normality of the data was tested by Shapiro-Wilk test. Data were tested by t-test or one-way analysis of variance followed by Tukey test as appropriate. Differences were considered significant at P<0.05. All statistical analyses were performed with GraphPad Prism (GraphPad Software, Boston, MA).

TABLE 1
Reagents used

| Reagent & Antibody | Company | Catalogue number |
|---|---|---|
| Rabbit polyclonal anti-Zic2 | Abcam | ab150404 |
| Goat polyclonal anti-Iba1 | Abcam | ab5076 |
| Rabbit polyclonal anti-CTMP | Cell Signaling | 4612s |
| | Technology | |
| Mouse anti-S100β (C-3) | Santa Cruz | sc-393919 |
| | Biotechnology | |
| Rabbit polyclonal anti-β-actin | Cell Signaling | 4967 |
| | Technology | |
| Donkey anti-goat IgG | ThermoFisher | A11055 |
| conjugated with Alexa | Scientific | |
| Fluor 488 | | |
| Goat anti-rabbit IgG-HRP | Santa Cruz | sc-2357 |
| | Biotechnology | |
| Mouse monoclonal anti-NeuN | Abcam | Ab104224 |
| Mouse monoclonal anti- | Invitrogen | 13-1500 (M13) |
| MAP2 | | |
| Rabbit polyclonal anti-CTMP | Abcam | ab106435 |
| (IF) | | |
| Donkey anti-rabbit IgG | ThermoFisher | A21207 |
| antibody conjugated with | Scientific | |
| Alexa Fluor 594 | | |
| Donkey anti-mouse IgG | ThermoFisher | A21202 |
| antibody conjugated with | Scientific | |
| Alexa Fluor 488 | | |
| Hoechst 33342 | ThermoFisher | 62249 |
| | Scientific | |
| DAPI | Biolegend | 422801 |
| Zic2-ORF cDNA (Rat) | Genecopoeia | EX-Rn12827-M02 |
| plasmid | | |
| pReceiver-M02 plasmid | Genecopoeia | EX-NEG-M02 |
| pGL3-enhancer vector | Promega | E1771 |
| Renilla luciferase vector | Promega | E2241 |
| pRL-TK | | |
| Lipofectamine LTX | ThermoFisher | A12621 |
| | Scientific | |
| CTMP-ORF | Genecopoeia | EX_Rn17554-M56_GS |
| M56-Vector | Genecopoeia | EX-NEG-M56 |
| EndoFectin Max transfection | Genecopoeia | EF013 |
| reagent | | |
| Geneticin | ThermoFisher | 11811023 |
| | Scientific | |
| Alexa Fluor 647 donkey anti- | Invitrogen | A31573 |
| rabbit IgG (H + L) | | |
| Rabbit polyclonal anti-Ki67 | Abcam | Ab15580 |
| Cell staining buffer | Biolegend | 420201 |
| 10 × RIPA buffer | ThermoFisher | 89901 |
| | Scientific | |
| Protease inhibitor cocktail | Sigma-Aldrich | P2714 |

### Results

### CTMP Induced Cell Senescence

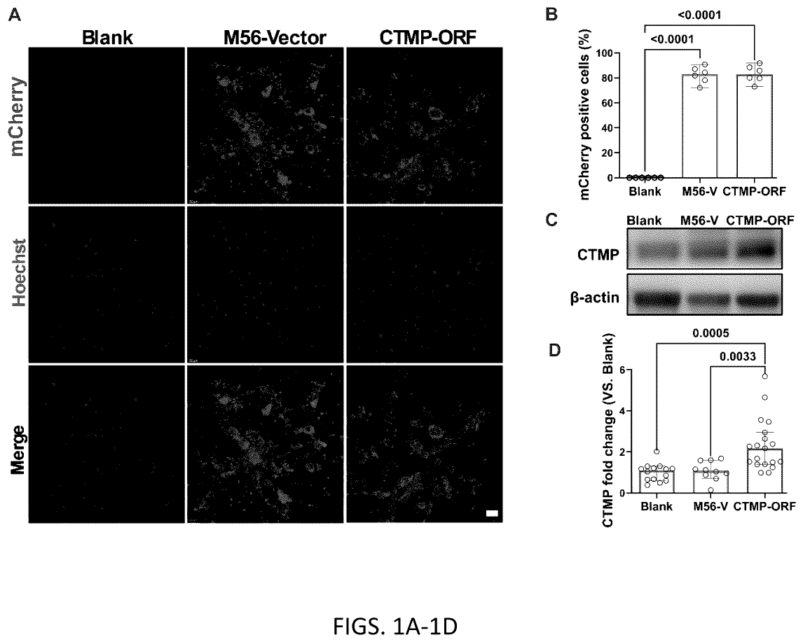

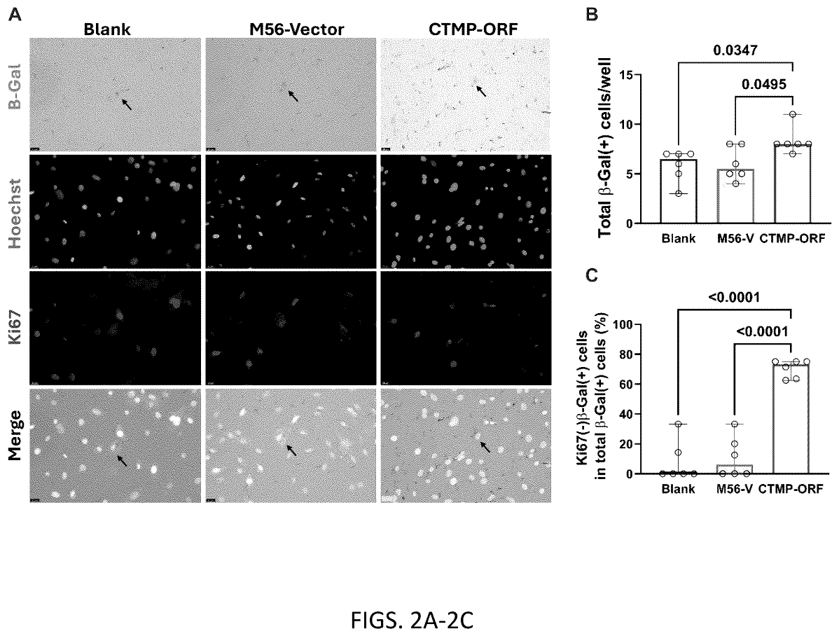

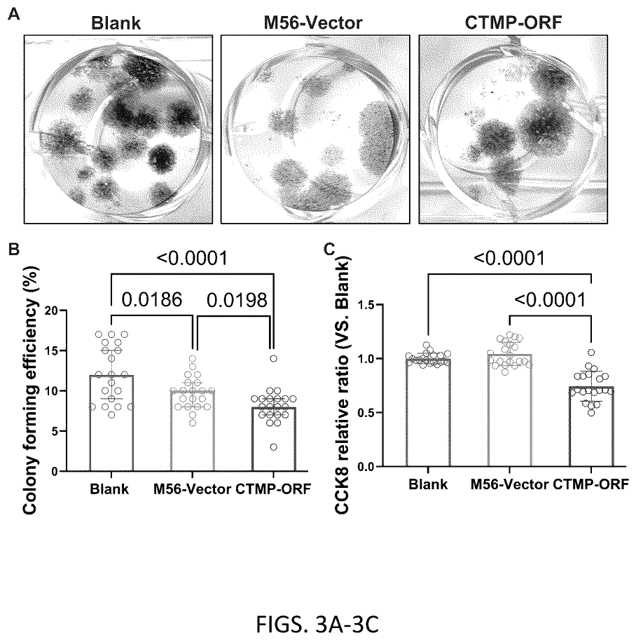

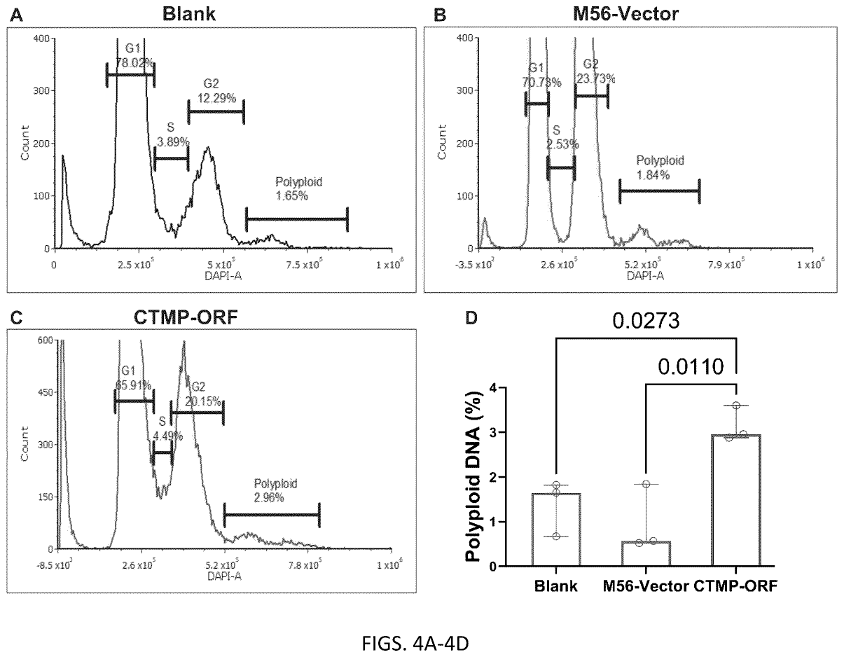

CTMP in the brain is increased with aging. Rat primary fibroblasts were transfected with a plasmid containing rat CTMP cDNA and mCherry code. The majority of cells were transfected by the plasmid as shown by the expression of mCherry (FIGS. 1A and 1B). These cells expressed more CTMP than control cells or cells transfected with empty plasmid (FIGS. 1C and 1D). This CTMP overexpression increased the number of cells expressing SA-β-Gal and the number of cells expressing SA-β-Gal but without the expression of Ki67 [Ki67 (−) SA-β-Gal (+) cells] (FIG. 2). Ki67 is a marker for cycling cells (Scholzen and Gerdes 2000). SA-β-Gal is a commonly used marker for cell senescence but can be increased by various factors including confluence stress and oxidative stress. These factors often increase Ki67. Thus, positive staining for SA-β-Gal but negative staining for Ki67 is considered a good indicator for cell senescence. There is an increase of senescence cells in cells expressing a high level of CTMP. CTMP overexpression decreased colony formation and cell proliferation, and increased percentage of cells with polyploid DNA (FIGS. 3 and 4), both are indicators of senescence. These results show that CTMP increase induces cell senescence.

CTMP Expression was Increased and Zic2 was Decreased with Aging

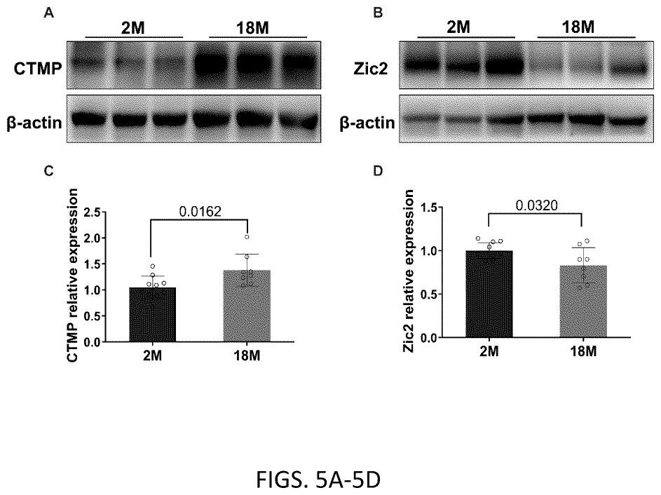

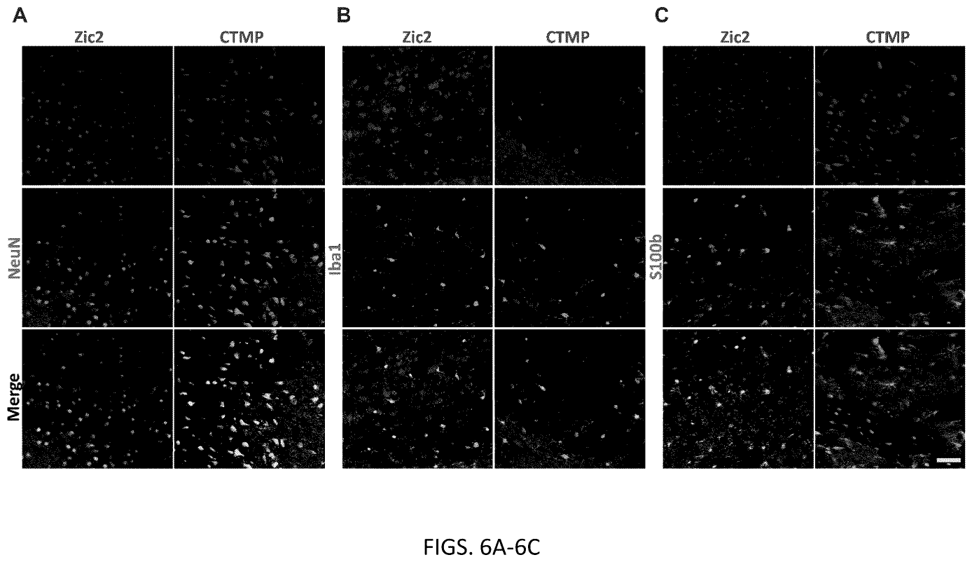

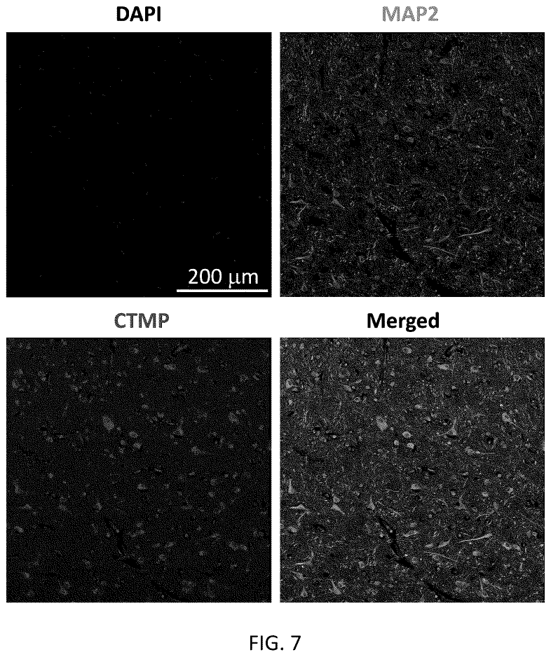

Western blotting analyses showed that CTMP was increased with aging in the cerebral cortex. Interestingly, Zic2 was decreased with aging in this brain region (FIG. 5). These results show a reciprocal expression pattern of CTMP and Zic2 in rat brain. CTMP and Zic2 were mostly expressed in the neurons but were observed in the astrocytes and microglia of the brain of Fischer 344 rats because CTMP and Zic2 were expressed in cells expressing neuronal nuclei (NeuN), S100β and ionized calcium binding adaptor molecule 1 (Iba-1) (FIG. 6), biomarkers of neurons, astrocytes and microglia, respectively. Similarly, CTMP staining was co-localized with the staining of microtubule-associated protein 2 (MAP2), a biomarker of neurons, in human cerebral cortex (FIG. 7), showing that CTMP is expressed in the neurons of human brain.

### Zic2 Directly Inhibited CTMP Expression

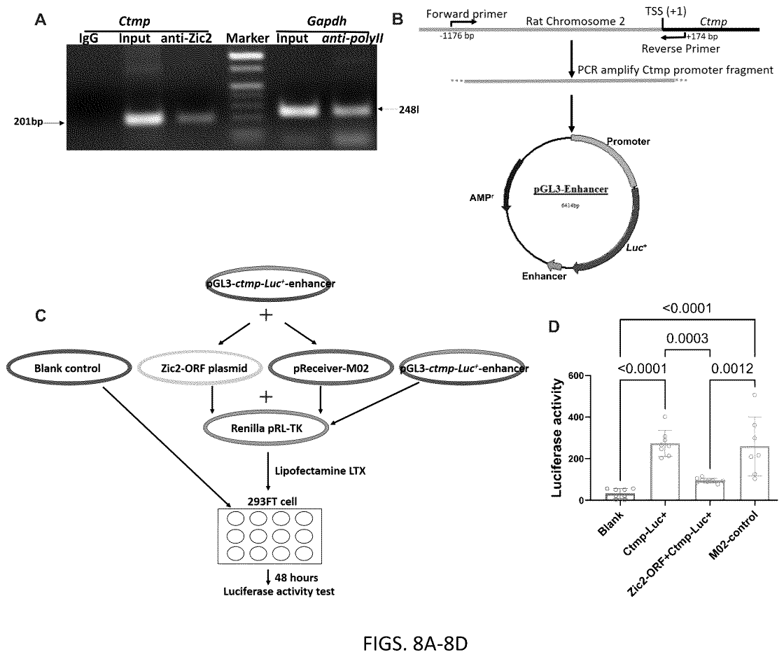

To determine whether Zic2 was involved in the regulation of CTMP expression, DNA samples prepared from 2-month old Fischer 344 rats were immunoprecipitated by an anti-Zic2 antibody. The immunoprecipates contained DNA fragments whose sequences were consistent with those of presumptive promoter regions of ctmp gene. As a negative control, non-specific IgG did not precipitate such DNA fragments. As a positive control, an anti-RNA polymerase II antibody precipitated DNA fragments of gapdh gene (FIG. 8A). Zic2 proteins bind the presumptive promoter regions of ctmp gene.

To determine whether the binding of Zic2 to ctmp gene affected the promoter activity, a plasmid that carried code for luciferase was constructed. The expression of luciferase was under the control of the preumptive promoter of the rat ctmp gene (FIG. 8B). The 293 FT cells transfected with this plasmid had increased luciferase activity. This activity was decreased by co-transfecting the cells with a M02 plasmid carrying code for Zic2. However, the plasmid that carried the backbone of the M02 plasmid but without the code for Zic2 did not affect the luciferase activity of cells transfected with the plasmid carrying code for luciferase whose expression was under the control of ctmp promoter (FIGS. 8C and 8D). These results show that Zic2 inhibits the activity of ctmp promoter.

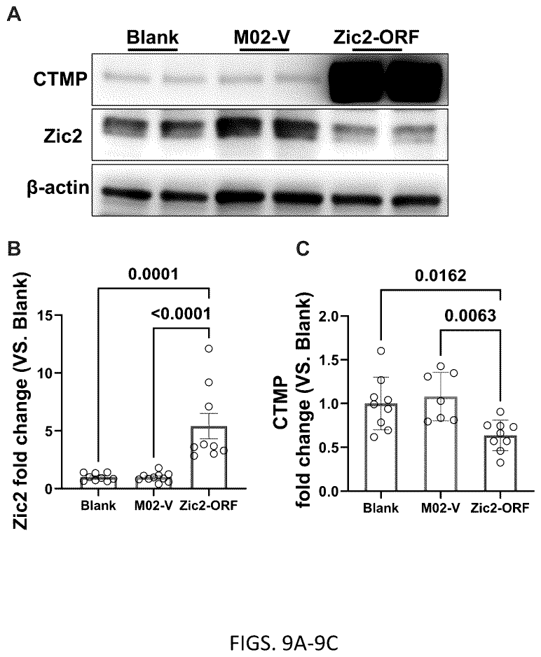

To determine whether Zic2 regulated CTMP expression in cells, 293 FT cells were transfected with a plasmid containing rat Zic2 cDNA. These cells had a very high level of Zic2 protein but a decrease in CTMP protein compared with control cells or cells that were transfected with empty plasmid (FIG. 9). These results, along with the results of Zic2 binding to the ctmp gene and the inhibition of Zic2 on the ctmp promoter activity, show that Zic2 can directly inhibit the expression of CTMP.

### Discussion

Various biological functions of CTMP have been revealed since it was discovered in 2001 (Li, Shan, and Zuo 2018; Maira et al. 2001; Miyawaki et al. 2009; Wang et al. 2014; Yu, Deng, and Zuo 2014). One important finding in this study is that CTMP may induce cell senescence. Cells expressing a high level of CTMP present multiple senescence biomarkers including SA-β-Gal expression but without the expression of Ki67, containing polypoid DNA, and a decrease in proliferation and colony formation. Cell senescence is a status of irreversible growth arrest of cells and is closely connected with aging (Kudlova, De Sanctis, and Hajduch 2022). Cell growth and proliferation are important in tissue repairing and regeneration and maintaining immunofunction. Thus, cell senescence will impair these functions. Various senolytic interventions have been proposed (Lucas, Cavadas, and Aveleira 2023). Our study has identified a novel molecular target, CTMP, to regulate cell senescence and reducing CTMP expression may be senolytic to improve tissue repairing and regeneration. On the other hand, cell senescence is beneficial to prevent cancer transformation (W. Huang et al. 2022). Thus, increasing CTMP may be a method to reduce cancer transformation of cells. Interestingly, CTMP is mainly expressed in the neurons in the brain of rats and humans. Mature neurons do not proliferate. The potential of CTMP in inducing cell senescence may contribute to the loss of proliferation ability in neurons.

Our previous study has shown a potential role of CTMP in the brain changes with aging (Li, Shan, and Zuo 2018). This current study has provided initial evidence that Zic2 expression is decreased in the brain with aging. More importantly, Zic2 is a direct suppressor for the expression of CTMP because Zic2 and CTMP had a reciprocal expression pattern in the brain with aging, Zic2 proteins bound the presumptive ctmp promoter region, Zic2 proteins inhibited ctmp promoter activity and over-expression of Zic2 decreased CTMP expression. These results suggest that the increase of CTMP in the brain with aging may be due to the decrease of Zic2. There was only one prior study that has investigated the transcription regulation of CTMP expression. That study showed that the neuroprotective effects of activating transcription factor 3 may be related to its inhibition of CTMP expression under hypoxic condition (C. Y. Huang et al. 2015).

Zic2 plays an important role in the development of central nervous system (Heeg-Truesdell and LaBonne 2006). Mutations in the Zic2 gene are associated with holoprosencephaly in humans (Brown et al. 1998; Warr et al. 2008). Zic2 increases neural progenitors via inhibiting the canonical Wnt/β-catenin signaling (Heeg-Truesdell and LaBonne 2006; Pourebrahim et al. 2011). Our results showed that the expression of Zic2 was decreased in the brain with aging. Zic2 existed in a very low level in the brain of old rats. Considering the previous findings of Zic2 on neural progenitors, the decreased Zic2 with aging may be a molecular mechanism for brain aging including decreased brain cell genesis in the old brain.

Our study was focused on determining the role of Zic2 in regulating CTMP expression with the primary focus on the brain. This regulating role shall occur in other tissues such as cancer cells and myocardium where CTMP has been found to regulate the growth of these cells (X. Liu et al. 2018; Y. P. Liu et al. 2013). Also, the regulation of Zic2 on CTMP expression appears to be direct because Zic2 binds the ctmp gene. However, indirect mechanisms such as via inhibiting the Wnt/β-catenin signaling cannot be excluded.

### REFERENCES

Alessio, N., Aprile, D., Cappabianca, S., Peluso, G., Di Bernardo, G., & Galderisi, U. 2021. “Different Stages of Quiescence, Senescence, and Cell Stress Identified by Molecular Algorithm Based on the Expression of Ki67, RPS6, and Beta-Galactosidase Activity.” International Journal of Molecular Sciences 22, no. 6
Brown, S. A., Warburton, D., Brown, L. Y., Yu, C. Y., Roeder, E. R., Stengel-Rutkowski, S., . . . . Muenke, M. 1998. “Holoprosencephaly due to mutations in ZIC2, a homologue of Drosophila odd-paired.” Nature Genetics 20, no. 2:180-183
Cassimeris, L., & Spittle, C. 2001. “Regulation of microtubule-associated proteins.” International Review of Cytology 210:163-226
Feng, C., Zhang, Y., Yin, J., Li, J., Abounader, R., & Zuo, Z. 2014. “Regulatory factor X1 is a new tumor suppressive transcription factor that acts via direct downregulation of CD44 in glioblastoma.” Neuro-Oncology 16, no. 8:1078-1085
Heeg-Truesdell, E., & LaBonne, C. 2006. “Neural induction in Xenopus requires inhibition of Wnt-beta-catenin signaling.” Developmental Biology 298, no. 1:71-86
Huang, C. Y., Chen, J. J., Wu, J. S., Tsai, H. D., Lin, H., Yan, Y. T., . . . . Lin, T. N. 2015. “Novel link of anti-apoptotic ATF3 with pro-apoptotic CTMP in the ischemic brain.” Molecular neurobiology 51, no. 2:543-557
Huang, W., Hickson, L. J., Eirin, A., Kirkland, J. L., & Lerman, L. O. 2022. “Cellular senescence: the good, the bad and the unknown.” Nature Reviews Nephrology 18, no. 10:611-627
Knobbe, C. B., Reifenberger, J., Blaschke, B., & Reifenberger, G. 2004. “Hypermethylation and transcriptional downregulation of the carboxyl-terminal modulator protein gene in glioblastomas.” Journal of the National Cancer Institute 96, no. 6:483-486
Kudlova, N., De Sanctis, J. B., & Hajduch, M. 2022. “Cellular Senescence: Molecular Targets, Biomarkers, and Senolytic Drugs.” International Journal of Molecular Sciences 23, no. 8
Lai, R. C., Shan, W. R., Zhou, D., Zeng, X. Q., Zuo, K., Pan, D. F., . . . . Zuo, Z. Y. 2019. “Sevoflurane promotes migration, invasion, and colony-forming ability of human glioblastoma cells possibly via increasing the expression of cell surface protein 44.” Acta Pharmacologica Sinica 40, no. 11:1424-1435
Li, J., Shan, W., & Zuo, Z. 2018. “Age-Related Upregulation of Carboxyl Terminal Modulator Protein Contributes to the Decreased Brain Ischemic Tolerance in Older Rats.” Molecular neurobiology 55, no. 7:6145-6154
Liu, X., Yang, Q., Zhu, L. H., Liu, J., Deng, K. Q., Zhu, X. Y., . . . . She, Z. G. 2018. “Carboxyl-Terminal Modulator Protein Ameliorates Pathological Cardiac Hypertrophy by Suppressing the Protein Kinase B Signaling Pathway.” Journal of the American Heart Association 7, no. 13
Liu, Y. P., Liao, W. C., Ger, L. P., Chen, J. C., Hsu, T. I., Lee, Y. C., . . . . Lu, P. J. 2013. “Carboxyl-terminal modulator protein positively regulates Akt phosphorylation and acts as an oncogenic driver in breast cancer.” Cancer Research 73, no. 20:6194-6205
Lucas, V., Cavadas, C., & Aveleira, C. A. 2023. “Cellular Senescence: From Mechanisms to Current Biomarkers and Senotherapies.” Pharmacological Reviews 75, no. 4:675-713
Maira, S. M., Galetic, I., Brazil, D. P., Kaech, S., Ingley, E., Thelen, M., & Hemmings, B. A. 2001. “Carboxyl-terminal modulator protein (CTMP), a negative regulator of PKB/Akt and v-Akt at the plasma membrane.” Science 294, no. 5541:374-380
Manning, B. D., & Toker, A. 2017. “AKT/PKB Signaling: Navigating the Network.” Cell 169, no. 3:381-405
Miyawaki, T., Ofengeim, D., Noh, K. M., Latuszek-Barrantes, A., Hemmings, B. A., Follenzi, A., & Zukin, R. S. 2009. “The endogenous inhibitor of Akt, CTMP, is critical to ischemia-induced neuronal death.” Nat Neurosci 12, no. 5:618-626
Pourebrahim, R., Houtmeyers, R., Ghogomu, S., Janssens, S., Thelie, A., Tran, H. T., . . . . Tejpar, S. 2011. “Transcription factor Zic2 inhibits Wnt/beta-catenin protein signaling.” Journal of Biological Chemistry 286, no. 43:37732-37740
Scholzen, T., & Gerdes, J. 2000. “The Ki-67 protein: from the known and the unknown.” Journal of Cellular Physiology 182, no. 3:311-322
Shan, W., Li, J., Xu, W., Li, H., & Zuo, Z. 2019. “Critical role of UQCRC1 in embryo survival, brain ischemic tolerance and normal cognition in mice.” Cellular & Molecular Life Sciences 76,
no. 7:1381-1396 Wang, Q., He, Y., Shen, Y., Zhang, Q., Chen, D., Zuo, C., . . . . Yu, Y. 2014. “Vitamin D inhibits COX-2 expression and inflammatory response by targeting thioesterase superfamily member 4.” Journal of Biological Chemistry 289, no. 17:11681-11694
Warr, N., Powles-Glover, N., Chappell, A., Robson, J., Norris, D., & Arkell, R. M. 2008. “Zic2-associated holoprosencephaly is caused by a transient defect in the organizer region during gastrulation.” Human Molecular Genetics 17, no. 19:2986-2996
Xu, X., Feng, J., & Zuo, Z. 2008. “Isoflurane preconditioning reduces the rat NR8383 macrophage injury induced by lipopolysaccharide and interferon gamma.” Anesthesiology 108, no. 4:643-650 Yang, N. C., & Hu, M. L. 2005. “The limitations and validities of senescence associated-beta-galactosidase activity as an aging marker for human foreskin fibroblast Hs68 cells.” Experimental Gerontology 40, no. 10:813-819
Yu, H., Deng, J., & Zuo, Z. 2014. “High-fat diet reduces neuroprotection of isoflurane post-treatment: Role of carboxyl-terminal modulator protein-Akt signaling.” Obesity (Silver Spring) 22, no. 11:2396-2405
Zeng, Q., Shan, W., Zhang, H., Yang, J., & Zuo, Z. 2021. “Paraventricular thalamic nucleus plays a critical role in consolation and anxious behaviors of familiar observers exposed to surgery mice.” Theranostics 11, no. 8:3813-3829
Zhang, J., Tan, H., Jiang, W., & Zuo, Z. 2014. “Amantadine alleviates postoperative cognitive dysfunction possibly by increasing glial cell line-derived neurotrophic factor in rats.” Anesthesiology 121, no. 4:773-785

### Example 2

### INTRODUCTION

Cell senescence represents a cell status of irreversible growth arrest and is associated with aging [1]. Our previous study has shown that carboxyl terminal modulator protein (CTMP) induces cell senescence in somatic cells of rats [2]. CTMP is an endogenous inhibitor of Akt, a kinase that is involved in many activities including cell survival and autophagy [3-7].

Autophagy is an important physiological process that maintains cellular homeostasis through lysosome-mediated protein degradation and organelle turnover [8]. Autophagy is decreased with aging [9-11]. Increasing autophagy attenuates the aging process and can increase life span. Inhibition of autophagy compromises the longevity-promoting effects of many interventions [9]. These lines of evidence support the role of decreased autophagy in aging. This role is conceivable as decreased autophagy impairs the clearance of damaged proteins and organelles, which ultimately leads to cell injury [9-11]. Obviously, there is a close relation between cell senescence and autophagy [12]. Importantly, autophagy is decreased in many neurodegenerative diseases including Alzheimer's disease (AD) [10]. This decrease is considered to be a significant factor for the accumulation of amyloid beta (AB), hyper-phospho-tau and other toxic substances in the brain [10]. Interestingly, defects in genes involved in autophagy have been found in patients with familial forms of neurodegenerative diseases [13]. These lines of evidence suggest the importance of autophagy impairment in neurodegenerative diseases.

A great breakthrough in biological and medical research in recent years is the demonstration of inducing human somatic cells into induced pluripotent stem cells (iPSCs). It has been shown that autophagy is important for the health of iPSCs-derived cells [14]. iPSCs have a high basal autophagy activity. Inhibiting autophagy induces iPSCs to die [15]. Thus, it is important to understand the regulation of autophagy in the iPSCs. Although healthy iPSCs may not present obvious signs of cell senescence, senescence can affect the potential of iPSCs for regenerative medicine and long-term culture possibility. In addition, little is known about the effects of autophagy on the senescence of iPSCs.

Based on the above information, this study was designed to determine whether CTMP induced senescence and affected autophagy in the iPSCs. To have human relevance, human iPSCs were used in the study. Control iPSCs and iPSCs with a gene mutation in the amyloid precursor protein (APP) gene whose existence leads to a form of familial AD were used to determine whether iPSCs with the mutation in the APP were vulnerable to senescence and the effects of CTMP on autophagy.

### Methods and Materials

Preparation of iPSCs Over-Expressing CTMP

Two human iPSC cell lines were purchased from the Jackson Laboratory (Bar Harbor, ME): control iPSCs (catalogue number: JIPSC001102) and AD iPSCs (catalogue number: JIPSC001086). The AD iPSCs carry the E693G mutation in the APP (APPE693G) gene. The control and AD iPSCs are isogenic cells with the only difference between the pair is the APPE693G mutation.

To over-express CTMP in human iPSCs, control and AD iPSCs were transduced with CTMP-open reading frame (CTMP-ORF) expression lentiviral particle (catalogue number: LPP-I1844-Lv224-200, Genecopoeia, Rockville, MD). A lentiviral vector without CTMP-ORF (catalogue number: LPP-NEG-Lv224-200, Genecopoeia) was used as control. Both lentiviral particles were constructed with a mCherry reporter. Transduction was performed by the protocol from Millipore Sigma (hESC Transduction Protocol) [16]. After transduction, cells were maintained in StemFlex Medium (catalogue number: A3349401, Thermo Fisher Scientific, Waltham, MA) containing 1 μg/ml puromycin thereafter. The transduction rate was determined by immunofluorescence staining and flow cytometry. The over-expression of CTMP protein in the CTMP-ORF lentivirus-transduced iPSCs were confirmed by Western blotting. Six groups of established iPSCs, control, control-V, control-CTMP, AD, AD-V and AD-CTMP, were used in the subsequent studies. The control-V and AD-V groups were iPSCs transduced with the lentivirus vector. The control-CTMP and AD-CTMP groups were iPSCs transduced with the lentivirus carrying CTMP-ORF.

### Western Blotting

The expressions of CTMP, microtubule-associated protein 1A/1B-light chain 3 (LC3)-I, LC3-II, P62, Beclin-1, P16 and mitochondrial PTEN-induced kinase 1 (PINK1) were determined by Western blotting. Antibodies used were: rabbit polyclonal anti-CTMP antibody (1:1000 dilution, catalogue number: 4612S, Cell signaling Technology, Danvers, MA), mouse monoclonal anti-LC3 antibody (1:1000 dilution, catalogue number: M186-3, MBL international Corporation, Carlsbad, CA), rabbit monoclonal anti-SQSTM1/P62 antibody (1:1000 dilution, catalogue number: M00300-1, Boster antibody and ELISA experts, Pleasanton, CA), rabbit monoclonal anti-beclin-1 (D40C5) antibody (1:1000 dilution, catalogue number: 3495s, Cell signaling Technology), rabbit monoclonal anti-p16 INK4A (D3W8G) antibody (1:1000 dilution, catalogue number: 92803S, Cell signaling Technology), rabbit monoclonal anti-PINK1 (D8G3) antibody (1:1000 dilution, catalogue number: 6946s, Cell signaling Technology), rabbit monoclonal anti-cytochrome c oxidase subunit IV (COX IV) (3E11) antibody (1:1000 dilution, catalogue number: 4850s, Cell Signaling Technology) and anti-β-actin antibody (1:1000 dilution, catalogue number: 4967, Cell Signaling Technology). Secondary antibody was mouse anti-rabbit (1:5000, catalogue number: sc-2357, Santa Cruz Biotechnology, Dallas, TX, USA) or mouse IgGk light chain binding protein (1:10000 dilution, catalogue number: sc-516102, Santa Cruz Biotechnology) conjugated with horseradish peroxidase. The results of corresponding protein bands were normalized to the values of β-actin, except in the case of LC3 I and II because the ratio of LC3 II and LC3 I was determined and PINK1 that was expressed in the mitochondria and was normalized by COX IV, a mitochondrial protein. The values of other experimental conditions were normalized by the results of control group in the same Western blotting gel.

### Colony Forming and Cell Counting

As we described before [2], colony forming ability was assessed by plating 100 cells/well on 6-well plates for two weeks. Cells were then fixed and stained with 1% crystal violet for 30 min at room temperature. Colonies with more than 50 cells were counted. The percentage of the number of colonies in the total number of cells seeded was calculated to reflect the colony-formation efficiency. Experiment was conducted three times with two replicate samples each time. Final comparison data was normalized to the values of control group.

Cell counting kit-8 (CCK-8, catalogue number: ab228554, Abcam, Waltham, MA) was used to determine cell proliferation ability. Briefly, 5000 cells/well were plated on 96-well plates for 48 h and then 10 μl water-soluble tetrazolium-8 solution was added to each well and incubated at 37° C. for 3 h before measuring the absorbance at 460 nm. Experiment was conducted six times with 4 to 6 replicate samples each time. The values of control group in each set of experiment were used to normalize the data of other experimental conditions. The data of different sets of experiments were pooled together for comparison among different experimental conditions.

### Flow Cytometry

As we performed before [2], cells were harvested and washed 2 times in cold phosphate buffered saline (PBS) and then re-suspended in 1.2 ml PBS (Ca2+ and Mg2+ free), and mixed with 3 ml ice cold 95% ethanol dropwise while being vortexed. Cells were fixed in the final 70% ethanol for at least 30 min at 4° C. Cells were washed twice with 12 ml PBS (centrifuged at 2000 rpm for 10 min) and then 1×106 cells were re-suspended in cell staining buffer (catalogue number: 420201, Biolegend, San Diego) containing 0.1 μg/ml 4′,6-diamidino-2-phenylindole (DAPI). The cells were incubated for 30 min on ice and then analyzed by a flow cytometer Attune NxT. The percentage of cells with polyploid DNA was measured. Experiment was conducted 4 times with 2 to 3 replicate samples each time. The values of control group were used to normalize the data of other experimental conditions.

### Statistical Analysis

Results are shown as mean±SD (parametric data with normal distribution) or median with 95% CI (non-parametric data and parametric data with non-normal distribution). The data of individual samples are included in the figures. The normality of the data was tested by the Shapiro-Wilk test. Data were tested by one-way analysis of variance followed by Tukey test as appropriate. Differences were considered significant at p<0.05. All statistical analyses were performed by using GraphPad Prism (GraphPad Software, Boston, MA).

### Results

Successful Establishment of iPSCs with Stable CTMP Over-Expression

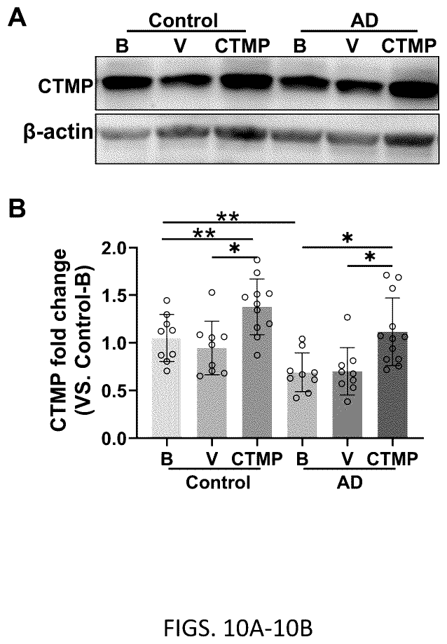

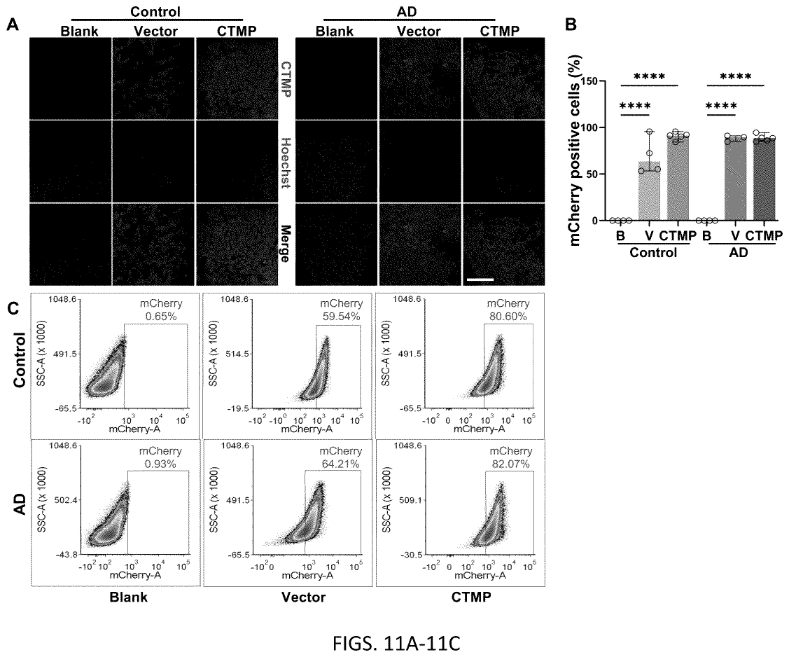

The human AD iPSCs had a lower level of CTMP than the control iPSCs at baseline. The transduction with lentiviral particles carrying the code for CTMP increased the level of CTMP in control and AD iPSCs, while transduction with lentiviral particles that did not carry code for CTMP did not change CTMP levels in the iPSCs (FIG. 10). To determine the transduction rate, the cells expressing mCherry whose code was carried in the lentivirus were measured by immunofluorescence staining and flow cytometry. The vast majority of these cells were positive for mCherry (FIG. 11). These findings confirmed the successful transduction of the lentiviral particles in the human iPSCs and the establishment of iPSCs stably over-expressing CTMP.

CTMP Induced Cell Senescence in iPSCs

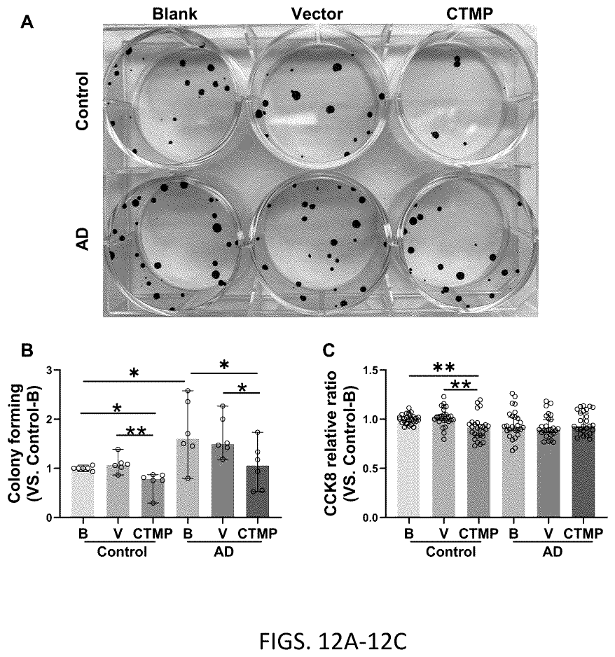

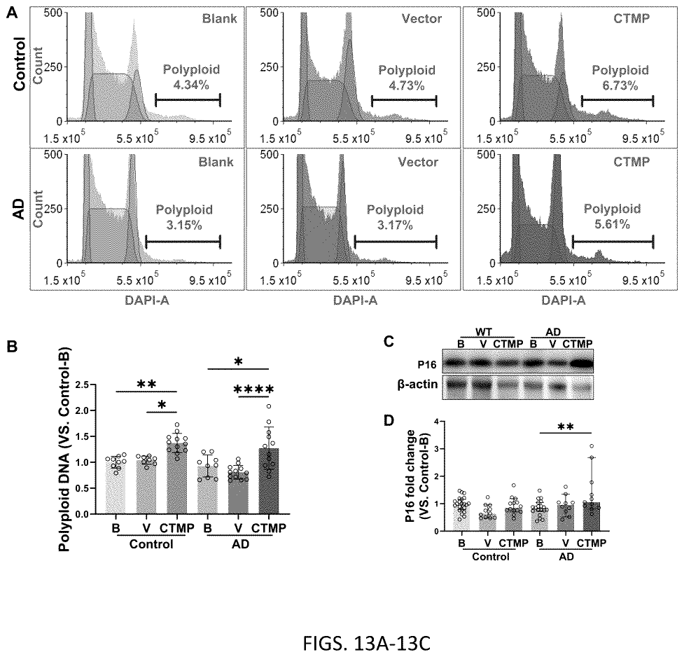

CTMP over-expression decreased the numbers of formed colonies in both control and AD iPSCs. The AD iPSCs that had a decreased CTMP level showed better colony-forming ability than control iPSCs (FIGS. 12A and 12B). These results suggest that CTMP inhibits cell survival. CTMP overexpression reduced the levels of CCK-8 assay in control iPSCs (FIG. 12C), indicating that CTMP over-expression reduces the viability and proliferation of these iPSCs. CTMP overexpression increased the number of cells with polypoid DNA in both control and AD iPSCs (FIGS. 13A and 13B). CTMP over-expression also increased the level of P16 in the AD iPSCs (FIG. 13C). Polypoid DNA and P16 are cell senescence biomarkers [2,17]. These results suggest that CTMP induces cell senescence in the control and AD iPSCs.

CTMP Reduced Autophagy in iPSCs

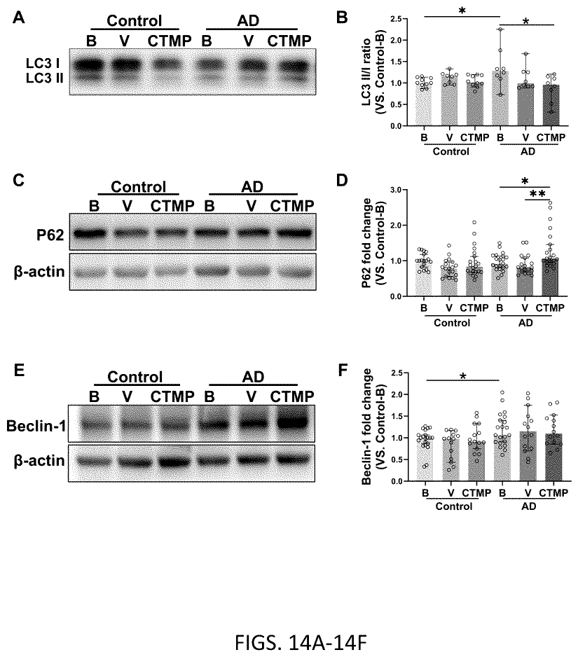

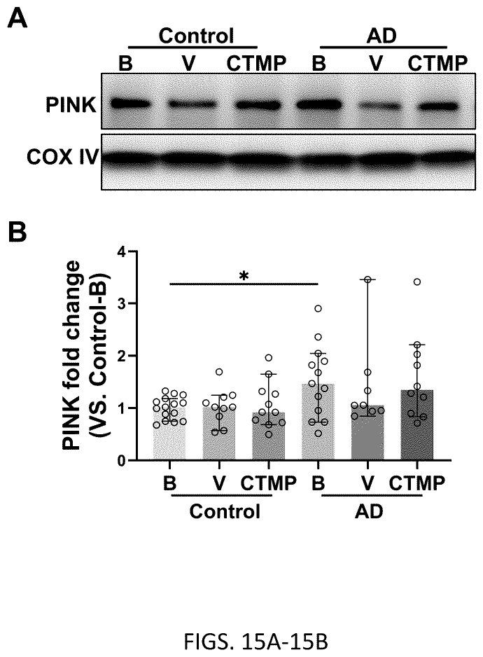

CTMP overexpression decreased the ratio of LC3II/I and increased P62 in the AD iPSCs. Also, the ratio of LC3II/I in the AD iPSCs was higher than those in the control iPSCs (FIGS. 14A-14D). These results suggest that CTMP inhibits autophagy in the AD iPSCs. Consistent with the changes in the levels of LC3 II vs. LC3 I, the levels of Beclin-1, an enzyme that is involved in macroautophagy [9-11], were increased in the AD iPSCs compared with control iPSCs (FIGS. 14E and 14F). The AD iPSCs had an increased level of PINK compared with control iPSCs (FIG. 15), suggesting that the mitochondria in the AD iPSCs are less healthy than those in the control iPSCs and that the mitophagy is increased in the AD iPSCs.

### Discussion

Our recent study has shown that CTMP can induce cell senescence in rodent cells and may be associated with brain aging in rats [2]. In this study, CTMP over-expression reduced the proliferation and survival ability and increased polyploid DNA in human control and AD iPSCs. Baseline CTMP expression was lower in the AD iPSCs, and CTMP overexpression increased P16 expression in human AD iPSCs. Since polyploid DNA and P16 are biomarkers for cell senescence [2,17], these results suggest that CTMP induces senescence in human iPSCs, extending our previous findings in rodents to human cells.

It has been shown that aging is associated with a decreased autophagy [9-11]. Interestingly, CTMP over-expression decreased the ratio of LC3 II to LC3 I and increased P62 in the AD iPSCs. Since a decreased ratio of LC3 II to LC3 I indicates a reduction of autophagosome and P62 is accumulated when autophagy is inhibited [9-11,18,19], our results suggest that CTMP decreases autophagy in these cells. Reduction of autophagy can lead to accumulation of misfolded protein and damaged organelles [8], which disturbs intracellular homeostasis. Thus, it is possible that the decreased autophagy may contribute to the senescence presentation in the AD iPSCs over-expressing CTMP. However, Beclin-1, an enzyme participating in the macroautophagy [9-11], and PINK1, a protein that is accumulated in unhealthy mitochondria to activate mitophagy [20], were not changed by CTMP over-expression, suggesting that Beclin-1 and mitophagy may not be the targets for CTMP to decrease autophagy in the human AD iPSCs.

The control and AD iPSCs used in our study are isogenic pairs of iPSCs. The mutation in the AD iPSCs causes increased amyloid-beta protofibrils that may contribute to the early onset of AD [21,22]. The isogenic pair setup reduces the confounding factors derived from different gene backgrounds and inter-individual variability if the control and AD iPSCs are not isogenic [23]. Interestingly, CTMP over-expression reduced the ratio of LC3 II to LC3 I and increased P62 and P16 in the AD iPSCs but not in the control iPSCs. These results suggest that the AD iPSCs are more sensitive than the control iPSCs for CTMP overexpression-induced inhibition of autophagy and cell senescence. Also, the AD iPSCs had a higher level of PINK1, suggesting that the AD iPSCs had more unhealthy mitochondria and possibly a higher mitophagy activity at baseline than the control iPSCs.

Our findings have significant implications. First, it appears that CTMP induces cell senescence in human cells and its over-expression may be detrimental to human iPSCs. Thus, CTMP may be a therapeutic target to keep iPSCs healthy. Second, our results suggest that the AD iPSCs are more vulnerable to detrimental stimulus and have more unhealthy mitochondria than control iPSCs, suggesting potential mechanisms for cell injury in patients with AD pathology. Finally, CTMP may regulate autophagy in human cells. Since abnormal autophagy contributes to many physiological and pathophysiological processes [9-11], regulating CTMP may be a possible intervention to correct these processes.

In summary, we have found that CTMP induces cell senescence in both control and AD iPSCs. CTMP inhibits autophagy in AD iPSCs, and these iPSCs may have unhealthy mitochondria. These findings suggest that regulating CTMP expression is a therapeutic intervention to reduce cell senescence in human cells.

### REFERENCES

1. Di Micco, R.; Krizhanovsky, V.; Baker, D.; d'Adda di Fagagna, F. Cellular senescence in ageing: From mechanisms to therapeutic opportunities. Nat. Rev. Mol. Cell. Biol. 2021, 22, 75-95, https://doi.org/10.1038/s41580-020-00314-w.
2. Shan, W.; Li, J.; Philpot, Z.; Zuo, Z. Carboxyl terminal modulator protein induces cell senescence and is upregulated with aging by zic2 in rats. J. Cell. Physiol. 2025, 240, e70007, https://doi.org/10.1002/jcp.70007.
3. Maira, S. M.; Galetic, I.; Brazil, D. P.; Kaech, S.; Ingley, E.; Thelen, M.; Hemmings, B. A. Carboxyl-terminal modulator protein (ctmp), a negative regulator of pkb/akt and v-akt at the plasma membrane. Science. 2001, 294, 374-380.
4. Miyawaki, T.; Ofengeim, D.; Noh, K. M.; Latuszek-Barrantes, A.; Hemmings, B. A.; Follenzi, A.; Zukin, R. S. The endogenous inhibitor of akt, ctmp, is critical to ischemia-induced neuronal death. Nat. Neurosci. 2009, 12, 618-626, https://doi.org/10.1038/nn.2299.
5 Li, J.; Shan, W.; Zuo, Z. Age-related upregulation of carboxyl terminal modulator protein contributes to the decreased brain ischemic tolerance in older rats. Mol. Neurobiol. 2017, https://doi.org/10.1007/s12035-017-0826-6.
6. Heras-Sandoval, D.; Perez-Rojas, J. M.; Hernandez-Damian, J.; Pedraza-Chaverri, J. The role of pi3k/akt/mtor pathway in the modulation of autophagy and the clearance of protein aggregates in neurodegeneration. 2014, 26, 2694-2701, Cell. Signal. https://doi.org/10.1016/j.cellsig.2014.08.019.
7. Rahman, M. A.; Rahman, M. D. H.; Mamun-Or-Rashid, A. N. M.; Hwang, H.; Chung, S.; Kim, B.; Rhim, H. Autophagy modulation in aggresome formation: Emerging implications and treatments of alzheimer's disease. Biomedicines. 2022, 10, https://doi.org/10.3390/biomedicines10051027.
8. Mizushima, N. Autophagy: Process and function. Genes. Dev. 2007, 21, 2861-2873, https://doi.org/10.1101/gad.1599207.
9. Rubinsztein, D. C.; Marino, G.; Kroemer, G. Autophagy and aging. Cell. 2011, 146, 682-695, https://doi.org/10.1016/j.cell.2011.07.030.
10. Kroemer, G. Autophagy: A druggable process that is deregulated in aging and human disease. J. Clin. Invest. 2015, 125, 1-4, https://doi.org/10.1172/JCI78652.
11. Dong, W.; Wang, R.; Ma, L. N.; Xu, B. L.; Zhang, J. S.; Zhao, Z. W.; Wang, Y. L.; Zhang, X. Autophagy involving age-related cognitive behavior and hippocampus injury is modulated by different caloric intake in mice. Int. J. Clin. Exp. Med. 2015, 8, 11843-11853.
12. Lee, Y.; Kim, J.; Kim, M. S.; Kwon, Y.; Shin, S.; Yi, H.; Kim, H.; Chang, M. J.; Chang, C. B.; Kang, S. B.; et al. Coordinate regulation of the senescent state by selective autophagy. Dev. Cell. 2021, 56, 1512-1525 e1517, https://doi.org/10.1016/j.devcel.2021.04.008.
13. Frake, R. A.; Ricketts, T.; Menzies, F. M.; Rubinsztein, D. C. Autophagy and neurodegeneration. J. Clin. Invest. 2015, 125, 65-74, https://doi.org/10.1172/JCI73944.
14. Hekman, K. E.; Koss, K. M.; Ivancic, D. Z.; He, C.; Wertheim, J. A. Autophagy enhances longevity of induced pluripotent stem cell-derived endothelium via mtor-independent ulk1 kinase. Stem. Cells. Transl. Med. 2022, 11, 1151-1164, https://doi.org/10.1093/stcltm/szac069.
15. Sotthibundhu, A.; McDonagh, K.; von Kriegsheim, A.; Garcia-Munoz, A.; Klawiter, A.; Thompson, K.; Chauhan, K. D.; Krawczyk, J.; McInerney, V.; Dockery, P.; et al. Rapamycin regulates autophagy and cell adhesion in induced pluripotent stem cells. Stem. Cell. Res. Ther. 2016, 7, 166, https://doi.org/10.1186/s13287-016-0425-x.
16. Ludwig, T. E.; Bergendahl, V.; Levenstein, M. E.; Yu, J.; Probasco, M. D.; Thomson, J. A. Feeder-independent culture of human embryonic stem cells. Nat. Methods. 2006, 3, 637-646, https://doi.org/10.1038/nmeth902.
17. Janzen, V.; Forkert, R.; Fleming, H. E.; Saito, Y.; Waring, M. T.; Dombkowski, D. M.; Cheng, T.; DePinho, R. A.; Sharpless, N. E.; Scadden, D. T. Stem-cell ageing modified by the cyclin-dependent kinase inhibitor p16ink4a. Nature. 2006, 443, 421-426, https://doi.org/10.1038/nature05159.
18. Bjorkoy, G.; Lamark, T.; Pankiv, S.; Overvatn, A.; Brech, A.; Johansen, T. Monitoring autophagic degradation of p62/sqstm1. Methods. Enzymol. 2009, 452, 181-197, https://doi.org/10.1016/S0076-6879 (08) 03612-4.
19. Fritzen, A. M.; Madsen, A. B.; Kleinert, M.; Treebak, J. T.; Lundsgaard, A. M.; Jensen, T. E.; Richter, E. A.; Wojtaszewski, J.; Kiens, B.; Frosig, C. Regulation of autophagy in human skeletal muscle: Effects of exercise, exercise training and insulin stimulation. J. Physiol. 2016, 594, 745-761, https://doi.org/10.1113/JP271405.
20. Jin, S. M.; Youle, R. J. Pink1- and parkin-mediated mitophagy at a glance. J. Cell. Sci. 2012, 125, 795-799, https://doi.org/10.1242/jcs.093849.
21. Basun, H.; Bogdanovic, N.; Ingelsson, M.; Almkvist, O.; Naslund, J.; Axelman, K.; Bird, T. D.; Nochlin, D.; Schellenberg, G. D.; Wahlund, L. O.; et al. Clinical and neuropathological features of the arctic app gene mutation causing early-onset alzheimer disease. Arch. Neurol. 2008, 65, 499-505, https://doi.org/10.1001/archneur.65.4.499.
22. Nilsberth, C.; Westlind-Danielsson, A.; Eckman, C. B.; Condron, M. M.; Axelman, K.; Forsell, C.; Stenh, C.; Luthman, J.; Teplow, D. B.; Younkin, S. G.; et al. The ‘arctic’ app mutation (e693g) causes alzheimer's disease by enhanced abeta protofibril formation. Nat. Neurosci. 2001, 4, 887-893, https://doi.org/10.1038/nn0901-887.
23. Ramos, D. M.; Skarnes, W. C.; Singleton, A. B.; Cookson, M. R.; Ward, M. E. Tackling neurodegenerative diseases with genomic engineering: A new stem cell initiative from the nih. Neuron. 2021, 109, 1080-1083, https://doi.org/10.1016/j.neuron.2021.03.022.
24. D'Alessandro, M. C. B.; Kanaan, S.; Geller, M.; Pratico, D.; Daher, J. P. L. Mitochondrial dysfunction in alzheimer's disease. Ageing. Res. Rev. 2025, 107, 102713, https://doi.org/10.1016/j.arr.2025.102713.

What is claimed is:

1. A method of preventing or treating a disease associated with cellular senescence in a subject in need thereof, the method comprising:
administering to the subject an effective amount of a CTMP inhibitor.

2. The method of claim 1, wherein the CTMP inhibitor comprises at least one of a polynucleotide sequence encoding Zic2, a CTMP gene silencing agent, or a small molecule compound that inhibits CTMP.

3. The method of claim 1, wherein the CTMP inhibitor reduces CTMP expression or activity.

4. The method of claim 1, wherein the disease associated with cellular senescence is selected from a cardiovascular disease, a metabolic disease, a musculoskeletal disease, a liver disease, a kidney disease, a lung disease, and a neurodegenerative disease.

5. The method of claim 4, wherein the disease is a neurodegenerative disease.

6. The method of claim 5, wherein the disease is Alzheimer's disease.

7. The method of claim 1, wherein the subject is a human.

8. A method for preventing or reducing senescence in a cell, the method comprising contacting the cell with a CTMP inhibitor.

9. The method of claim 8, wherein the CTMP inhibitor comprises at least one of a polynucleotide sequence encoding Zic2, a CTMP gene silencing agent, or a small molecule compound that inhibits CTMP.

10. The method of claim 9, wherein the polynucleotide encoding Zic2 is packaged in a vector.

11. The method of claim 10, wherein the vector is an M02 vector.

12. The method of claim 8, wherein the cell is an iPSC.

13. The method of claim 8, wherein the cell is derived from a subject having an age-related disease.

14. The method of claim 13, wherein the age-related disease is Alzheimer's disease.


SEQUENCE LISTING (10 sequences)

SEQ ID NO: 1 - DNA, 20 bp, synthetic construct
  source 1..20: mol_type = other DNA
ggctaactctatccaatcca

SEQ ID NO: 2 - DNA, 18 bp, synthetic construct
  source 1..18: mol_type = other DNA
gtaggtcactcgcacatt

SEQ ID NO: 3 - DNA, 20 bp, synthetic construct
  source 1..20: mol_type = other DNA
tcactaccgaagaacaacga

SEQ ID NO: 4 - DNA, 21 bp, synthetic construct
  source 1..21: mol_type = other DNA
ggataggactcagggaataca

SEQ ID NO: 5 - DNA, 29 bp, synthetic construct
  source 1..29: mol_type = other DNA
tccggtaccatgttccagattagcaacct

SEQ ID NO: 6 - DNA, 29 bp, synthetic construct
  source 1..29: mol_type = other DNA
ggcaagcttcaatgtcctctcaaccagaa

SEQ ID NO: 7 - DNA, 2903 bp, synthetic construct
  source 1..2903: mol_type = other DNA
cgcgcctcctccgccgccgcctcctcttcctctccgcgccttcgctcggcgcccggccgcctgaggcagatacaggcggcggcggccggcgcagtagcccggctcccgaggctgcagtcgccagtgtttccaccgccggttcgcggagcccggcgtccagaggacgcgcctcgccgatcaggaggggaaatccaggcagcagggctcaccggggccggcggggcggccgggcgcgctggccatgcttctggacgcggggccgcagttcccggccatcggggtgggcagcttcgcgcgccaccaccaccactcggccgcggcagcggcggctgcggcggccgagatgcaggaccgggagctgagcctggcggcggcacagaacggcttcgtggactcggccgcagcgcacatgggcgccttcaagctcaaccccggggcacacgaactgtctcctggtcagagttcggcgttcacgtcgcaaggtccgggcgcctacccgggttcggctgcagcggccgctgcagccgcggctctagggccccacgccgctcacgttggctcttattcggggcctccctttaattccacccgggacttcctgttccgtagccggggcttcggggactcggcgccgggaggcggccagcacgggctcttcggacccggcgccggcggcctccaccatgcgcactcggacgcgcagggccaccttctcttccctggcctcccggaacagcacgggccgcacgcctcgcagaacgtgctcaatgggcaaatgcgcctagggctgccgggcgaagtgttcgggcgctcagagcaataccgccaagtggccagcccgcggaccgacccttactcggcggcgcagctccacaaccagtacggccctatgaatatgaacatggggatgaacatggcagcggccgcagcccaccaccaccaccaccatcaccaccctggtgcctttttccgctacatgcggcagcagtgcatcaagcaagagctcatctgcaagtggatcgacccggagcagctaagcaatcccaagaaaagctgcaacaaaactttcagcaccatgcacgagctggtgacccacgtctctgtagagcatgtcggcggcccggaacagagcaaccacgtctgcttctgggaggagtgtccacgcgagggcaaaccctttaaggccaaatacaaactggtcaaccacatccgcgtgcacacgggcgagaaacccttcccttgtcctttcccgggttgtggcaaggtcttcgcacgctccgagaacctcaagatccacaaaagaactcacacaggggagaaacctttccagtgtgagttcgagggctgtgaccgacgcttcgccaacagcagcgacaggaagaagcacatgcacgtccacacctcagataagccctatctctgcaagatgtgcgacaagtcctacacgcatcccagctccttgcggaagcacatgaaggtccatgagtcctcccctcagggctccgagtcctccccggctgccagctctggctacgagtcgtccacacccccagggttggtgtcccccagcgcagagccacaaagcagctccaacctgtccccagcagcggcagcggcggcggcagcggctgcagcagcggcggccgcggtgtccgcagtgcaccgaggcgcgggttctggcagtagcggctccggagggggctcggcggccggcagcggtgggggcggcggcggggcgggcggcgggggcggcggcagctctggcgggggcagcgggacagccggaggccatagcggcctctcctccaacttcaatgaatggtacgtgtgaagggccaggcctttctcccattccctgttcccttatccaccgtcgccctcccaaaaacccaccgagggcaccttaggatcatcttactaaaattatgagtctgatttttatggtgaaaaaaattttaccagcagaaggatttttaaaagtttttttttctttttaaagaaaacaatctaggcatgaaaagcaacatctctctgtatctctgaagctgaaaacataaaaaaaattttttgaagaaagtaaactccacaaaaatatcgagccaaacttccctagcaacccccagcccactttccttcccatatgcttcccagtctttttgacaaactgtacatagcggactccttccttttccacggtgattttaatggcttattggtggatagaagtttgtccatttgtaaactccggattgcgttccctcctgcctttctcccattccccttccccaagtgacggaccttttcttttttccttttagtttacccagtctttttttttttaaagtaatgtggaagaaaatggtttattttgtattgtggtattaaatattgtgttccttttaaatgaggcaacttgattgtaaacttcatgcgactatagactggaaaatatgagccgtgccaaagtttcccttctgtttcttcaacacaccaacccatagcacacatcaccaccaccaccaacgcttgtgaatgtatttttctgttagctgggtttacatgtgatgtttcagtgcttttgcgagttcgatttgttagttcttgtatgaaagtttgggggtggggtgggtgggagtaaacgttgtgccgttagctttttccgaaataacacccttctgtaaatatccgttgccatatttatccatttgtaattaaattatggtattaacttgctacagaggaaacaatatttataaagaatgtttcttaactataaatatgtacaattgtgggcataaactgtttcagatttttttatttgaaggtttaagtggtttgatcatttcttgtgatgttttgagagtaatgcatacagaaatataataaaatgtgttgaaactgca

SEQ ID NO: 8 - DNA, 1756 bp, synthetic construct
  source 1..1756: mol_type = other DNA
gtccggaatccgcgtgccgggtagtgtggctctttctggttgagaggacattgcagcgtcctgccctcggcttgaacgatgttgagaagctgcgctatgcgcctgcgcactcttggggccacgcctgcaaggcggcctgaagcaacgaggaggttattttcttctgaggaagtcgtctgtaaggactatgctctccctaaccccagctggactaaggacctaagactcctttttgaccagttcatgaagaaatgtgaagatggctcctggaaacgtttgccttcatacagacaaaaccctcctcaagcccttcaagagttccaaacccactttgttgactcaaagtttaagaaagaagaacaaatgtcaaaggcacaacagttcaccagaagcttagaagaaggactaggctttgagtacgcgatgttctataataaggctgagaagagaattgtctgcttgtttcaaggaggtcttcacctgcaaggaatgcctggatttgttcatggaggtgccattgcaaccatcatcgacatcactgccggcatgtgtgcattttctgagggcattgtcatgactgccaatctcaacatcgactataaaaaacctatcccccttctttctgttgttgtggtaaatagccaacttcaaaaaattgaaggaaggaaactttttgtttcctgtactattcaaagtactgatgagaagactttacatacacaggcaacagccttatttataaagctggatcctgacaaacctttgacataaaagaactctggtgagttccacaccgttctgcatgaggtccccgccccctccagaagtagcacctatctccactcctgtcacagctgctgaatcccccagggaaagtctgctctcacagctccttcagcgcagcctctgacgtggctgcaggaactcactctcaccttccagagttatcataagaagtactgcccgggggcgtctcagcagtaatctgtatgtccacgtgacaactgtgtggttctaactcgtgaacagacttcaaagctaacaagcataggacctgctctgctgtgggctgcagaaaaaaacaaaagaccatattgagacttgggctgcaaatgtggaaatgcacgaaaaccacaggcagggatagatccacagagcgttgagtgacgtgcgtgtgtgcacaagccgcgccgggtcttacagaactaagtttgttgtatctgaacaaactctctttggccacatcaggaacacagctcattgctgtgggaatggcccccacacatcattgtctggggccctagagattaaaatactgttcatttttctggcctgtgctgctgtagcttagaacactgtagtctgtcacccctggaagaaggctttgttacatttctgggactgtaatgaagagctacaagctgggtggtttaaacaaataaaaaacgtcactggcacagccccaggggccagcagtctaaaagtattaacaaaacgttgcttcttaaggttctgggcgggcactttcacatctctcctggcttctaccaaggaagcatccagctttccttggtttttagacacagcataattcctaattgcatctcttttatcatcttctctcggttgtatccacatttccctttgtgaatgtttatttatttatttgtctgggtgggtgtgtgtgttcaggtgctaaaataaattgggagattagtgttctacaaagtgaaaaaaaaaaaaaaaaaaaaaaaaa

SEQ ID NO: 9 - protein, 532 aa, synthetic construct
MLLDAGPQFPAIGVGSFARHHHHSAAAAAAAAAEMQDRELSLAAAQNGFVDSAAAHMGAFKLNPGAHELSPGQSSAFTSQGPGAYPGSAAAAAAAAALGPHAAHVGSYSGPPFNSTRDFLFRSRGFGDSAPGGGQHGLFGPGAGGLHHAHSDAQGHLLFPGLPEQHGPHGSQNVLNGQMRLGLPGEVFGRSEQYRQVASPRTDPYSAAQLHNQYGPMNMNMGMNMAAAAAHHHHHHHHHPGAFFRYMRQQCIKQELICKWIDPEQLSNPKKSCNKTFSTMHELVTHVSVEHVGGPEQSNHVCFWEECPREGKPFKAKYKLVNHIRVHTGEKPFPCPFPGCGKVFARSENLKIHKRTHTGEKPFQCEFEGCDRRFANSSDRKKHMHVHTSDKPYLCKMCDKSYTHPSSLRKHMKVHESSPQGSESSPAASSGYESSTPPGLVSPSAEPQSSSNLSPAAAAAAAAAAAAAAAVSAVHRGGGSGSGGAGGGSGGGSGSGGGGGGAGGGGGGSSGGGSGTAGGHSGLSSNFNEWYV

SEQ ID NO: 10 - protein, 107 aa, synthetic construct [sequence omitted]